(19) United States
(12) Patent Application Publication
(10) Pub. No.: US 2011/0268684 A1
(43) Pub. Date: Nov. 3, 2011

(54) HAIR CONDITIONERS COMPRISING IMIDAZOLINES AND ESTER OILS

(76) Inventors: Marlene Battermann, Asendorf (DE); Dieter Goddinger, Klein Nordende (DE); Tanja Bösel, Langenfeld (DE); Christa Hartwich, Elmshorn (DE); Nicole Fernandes, Hamburg (DE)

(21) Appl. No.: 13/153,607

(22) Filed: Jun. 6, 2011

Related U.S. Application Data
(63) Continuation of application No. PCT/EP2009/065282, filed on Nov. 17, 2009.

(30) Foreign Application Priority Data
Dec. 3, 2008 (DE) 10 2008 060 147.0

(51) Int. Cl. A61K 8/92; A61Q 19/00; A61Q 5/12

(52) U.S. Cl. 424/70.11; 424/70.1; 514/396

(57) ABSTRACT

Cosmetic preparations, in particular hair conditioners, contain at least one imidazoline derivative including at least two long fat groups, and at least two ester oils.

### CROSS-REFERENCES TO RELATED APPLICATIONS

This application is a continuation of PCT Application Serial No. PCT/EP2009/065282, filed on Nov. 17, 2009, which claims priority under 35 U.S.C. §119 to DE 10 2008 060 147.0, filed on Dec. 3, 2008. Both of PCT/EP2009/058789 and DE 10 2008 038 479 are hereby incorporated by reference in their entirety.

### FIELD OF THE INVENTION

The present invention generally relates to hair treatment agents containing cationic imidazolines and to the use of said agents to treat skin and hair.

### BACKGROUND OF THE INVENTION

Severe stress is imposed on hair as a result of environmental stresses and hair care procedures such as dyeing, permanent waves, and cleaning with shampoos. Consequently, there is an increasing need and importance for hair care products having a maximally long-lasting effect.

Usual hair care compositions contain silicones, which may have the adverse affect of completely or partially impeding penetration of many active substances into the interior of the hair. In addition, during hair styling a composition having silicone has a burdensome and complicating effect on styling. A high-volume hairstyle that is easy to comb with both the wet and dry hair is often not obtainable with these compositions.

Quaternary ammonium compounds of the mono-, di-, and/or trialkylammonium compound types have been known for some time. One disadvantage of these compounds, however, is their lack of biodegradability. Cationic compounds containing at least one ester group (the so-called esterquats) were therefore developed. These exhibit, however, in terms of the softness and feel of wet skin and wet hair, as well as the softness and feel of the skin or hair once it has dried after washing, a feel that is judged to be unpleasantly dull, and is also perceived as audibly “squeaky.”

A need therefore still exists for active substances or active-substance combinations for cosmetic agents that have good hair care-providing properties, good biodegradability, and are free of silicones.

Furthermore, other desirable features and characteristics of the present invention will become apparent from the subsequent detailed description of the invention and the appended claims, taken in conjunction with the accompanying drawings and this background of the invention.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Surprisingly, it has been found that cationic imidazolines of formula Ia, in particular those that have a residue R chain length of at least 20 carbon atoms, and particularly preferably 21 carbon atoms, in combination with at least two cosmetic ester oils, satisfy in outstanding fashion the objects discussed previously for hair-care-providing compositions.

In a preferred embodiment, at least one further cationic compound is contained. This further cationic compound is selected from the cationic imidazolines of formula Ib having a residue R chain length from 8 to 18 carbon atoms, the esterquats, the cationic compounds of formula (Tkat2), or the amidoamines and/or the quaternized amidoamines or the cationic polymeric compounds. The obligatorily present ester oils are particularly preferably selected from the esters of carbonic acid with fatty alcohols, in particular selected from the symmetrical esters of carbonic acid with fatty alcohols and/or from the esters of fatty acids with fatty alcohols and/or an ester oil of the structural formula (D4-II). The embodiments preferred according to the present invention contain at least one physiologically acceptable cosmetic carrier and furthermore contain the following ingredients as presented in the respective embodiment. In no case, however, is a silicone contained.

### Embodiment 1

at least one imidazoline derivative of formula Ia,
at least two ester oils,
at least one amine and/or cationized amine, in particular at least one amidoamine and/or cationized amidoamine

### Embodiment 2

at least one imidazoline derivative of formula Ia,
at least two ester oils,
at least one imidazoline derivative of formula Ib

### Embodiment 3

at least one imidazoline derivative of formula Ia,
at least two ester oils,
at least one esterquat

### Embodiment 4

at least one imidazoline derivative of formula Ia,
at least two ester oils,
at least one cationic surfactant of formula (Tkat2)

### Embodiment 5

at least one imidazoline derivative of formula Ia,
at least two ester oils,
at least one amine and/or cationized amine, in particular at least one amidoamine and/or cationized amidoamine,
at least one imidazoline derivative of formula Ib

### Embodiment 6

at least one imidazoline derivative of formula Ia,
at least two ester oils,
at least one amine and/or cationized amine, in particular at least one amidoamine and/or cationized amidoamine,
at least one esterquat

### Embodiment 7

at least one imidazoline derivative of formula Ia,
at least two ester oils,
at least one amine and/or cationized amine, in particular at least one amidoamine and/or cationized amidoamine,
at least one cationic surfactant of formula (Tkat2)

### Embodiment 8

at least one imidazoline derivative of formula Ia,
at least two ester oils,
at least one imidazoline derivative of formula Ib,
at least one esterquat

### Embodiment 9

at least one imidazoline derivative of formula Ia,
at least two ester oils,
at least one imidazoline derivative of formula Ib,
at least one cationic surfactant of formula (Tkat2)

### Embodiment 10

at least one imidazoline derivative of formula Ia,
at least two ester oils,
at least one cationically charged polymeric compound,
at least one esterquat,
at least one cationic compound of formula (Tkat2)

### Embodiment 11

at least one imidazoline derivative of formula Ia,
at least two ester oils,
at least one esterquat,
at least one amine and/or cationized amine, in particular at least one amidoamine and/or cationized amidoamine,
at least one cationic surfactant of formula (Tkat2)

### Embodiment 12

at least one imidazoline derivative of formula Ia,
at least two ester oils,
at least one esterquat,
at least one amine and/or cationized amine, in particular at least one amidoamine and/or cationized amidoamine,
at least imidazoline derivative of formula Ib

### Embodiment 13

at least one imidazoline derivative of formula Ia,
at least two ester oils,
at least one cationic surfactant of formula (Tkat2),
at least one amine and/or cationized amine, in particular at least one amidoamine and/or cationized amidoamine,
at least one imidazoline derivative of formula Ib

### Embodiment 14

at least one imidazoline derivative of formula Ia,
at least two ester oils,
at least one esterquat,
at least one amine and/or cationized amine, in particular at least one amidoamine and/or cationized amidoamine,
at least one cationic surfactant of formula (Tkat2),
at least one imidazoline derivative of formula Ib.

Particularly preferably, the embodiments recited above contain at least three ester oils. It is furthermore highly preferred if, in the aforementioned embodiments, not only are at least three ester oils used but moreover at least one cationically charged polymeric compound is contained.

The use of these combinations results in surprisingly good properties of the treated skin and hair that considerably exceed the effects of comparable compositions having silicone. The compositions according to the present invention have all the advantages of silicone-containing compositions but without exhibiting their great disadvantages.

A first subject of the present invention is therefore a composition for treating keratinic fibers, containing
a) at least 0.01 wt % of a cationic imidazoline derivative having at least two long fat residues in accordance with formula Ia,
b) at least two ester oils,
c) a cosmetic carrier, and
d) no silicone.

The agents according to the present invention contain an active-substance combination of at least two constituents, constituents a) and b) being used within a specific weight ratio with respect to each other. In preferred agents according to the present invention, the weight ratio of imidazoline derivatives a) of formula Ia to one of the further cationically charged polymeric compounds b) is 50:1 to 1:50, preferably 20:1 to 1:20, particularly preferably 10:1 to 1:10, especially preferably 5:1 to 1:5. These ratios are also applicable, in the embodiments according to the present invention having at least one further cationic surfactant, with regard to the sum of all the cationic surfactants contained. In the embodiments according to the present invention in which more than two further cationic surfactants are contained, it is preferred according to the present invention that these ratios apply exclusively with regard to the imidazoline derivative of formula Ia. In this particular case, it is most highly preferred if the weight ratio of constituents a) to b) is between 3:1 and 1:3.

Ingredients a) and b) are described below in detail. When reference is made hereinafter to active-substance complex (A), this statement refers to the ingredients a) and b) obligatorily contained in the agents according to the present invention.

“Hair treatment agents” for purposes of the present invention are, for example, hair dyeing agents, hair bleaching agents, hair shampoos, hair conditioners, conditioning shampoos, hair sprays, hair rinses, hair therapies, hair packs, hair tonics, permanent-wave fixing solutions, hair dyeing shampoos, hair coloring agents, hair-setting agents, hair setting compositions, hair styling preparations, blow-dry wave lotions, setting foams, hair gels, hair waxes, or combinations thereof.

“Combability” is understood according to the present invention as both the combability of the wet fibers and the combability of the dry fibers. The combing work expended, or the force expended, during the operation of combing an assemblage of fibers serves as an indication of combability. The measurement parameters can be assessed in sensory fashion by one skilled in the art, or quantified using measurement devices.

“Softness” is defined as the tactility of an assemblage of fibers, in which context the person skilled in the art feels and evaluates the “fullness” and “suppleness” parameters of the assemblage.

“Shaping” is understood as the ability to impart a change in shape to an assemblage of previously treated keratin-containing fibers, in particular human hairs The term “stylability” is also used in hair cosmetics.

An “oxidizing hair treatment” is defined according to the present invention as the action of an oxidizing cosmetic agent, containing at least one oxidizing agent in a cosmetic carrier, on hair.

Suitable according to the present invention as cosmetic carriers are, in particular, O/W, W/O, and W/O/W emulsions in the form of creams or gels or also surfactant-containing foaming solutions such as, for example, shampoos, foam aerosols or other preparations, that are suitable in particular for application to the hair. It is also conceivable, however, to integrate the ingredients into a powdered or even tablet-shaped formulation that is dissolved in water prior to utilization. The cosmetic carriers can be, in particular, aqueous or aqueous alcoholic.

An “aqueous” cosmetic carrier contains at least 50 wt % water.

“Aqueous alcoholic” cosmetic carriers are to be understood for purposes of the present invention as aqueous solutions containing 3 to 70 wt % of a C1 to C6 alcohol, in particular methanol, ethanol, or propanol, isopropanol, butanol, isobutanol, tert-butanol, n-pentanol, isopentanols, n-hexanol, isohexanols, glycol, glycerol, 1,2-pentanediol, 1,5-pentanediol, 1,2-hexanediol, or 1,6-hexanediol. The agents according to the present invention can additionally contain further organic solvents such as, for example, methoxybutanol, benzyl alcohol, ethyl diglycol, or 1,2-propylene glycol. All water-soluble organic solvents are preferred in this context.

The agents according to the present invention contain as ingredient a) at least one quaternary imidazoline compound, i.e. a compound that comprises a positively charged imidazoline ring. Formula I depicted below shows the structure of these compounds.

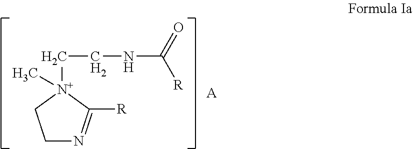

The residues R each denote, mutually independently, a saturated or unsaturated, linear or branched hydrocarbon residue having a chain length of 20 to 30 carbon atoms. The preferred compounds of formula I contain the same hydrocarbon residue for each residue R. The chain length of the residues R is at least 20 carbon atoms. Compounds having a chain length of at least 21 carbon atoms are preferred. “A” denotes a physiologically acceptable anion. Encompassed according to the present invention as an anionic counter ion are halides, for example fluoride, chloride, or bromide, alkyl sulfates such as methosulfate or ethosulfate, phosphates, citrate, tartrate, maleate, or fumarate. A commercial product of this chain length is known, for example, under the designation Quatemium-91.

The imidazolines of formula Ia are contained in the compositions according to the present invention in quantities from 0.01 to 20 wt %, preferably in quantities from 0.01 to 10 wt %, and very particularly preferably in quantities from 0.1 to 7.5 wt %. The best results of all are obtained in this context with quantities from 0.1 to 5 wt %, based in each case on the total composition of the respective agent.

Ester oils are used as ingredient b). The ester oils are defined as follows:

“Ester oils” are to be understood as the esters of C6 to C30 fatty acids with C2 to C30 fatty alcohols. The monoesters of fatty acids with alcohols having 2 to 24 carbon atoms are preferred. Examples of fatty-acid components used in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, as well as industrial mixtures thereof. Examples of the fatty-alcohol components in the ester oils are isopropyl alcohol, capronyl alcohol, capryl alcohol, 2-ethylhexyl alcohol, caprinyl alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol, and brassidyl alcohol, as well as industrial mixtures thereof.

Isopropyl myristate (Rilanit® IPM), isononanoic acid C16-18 alkyl esters (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol A), di-n-butyl adipate (Cetiol B), myristyl myristate (Cetiol MM), cetearyl isononanoate (Cetiol® SN), oleic acid decyl ester (Cetiol® V) are particularly preferred according to the present invention.

The ester oils can of course also be alkoxylated with ethylene oxide, propylene oxide, or mixtures of ethylene oxide and propylene oxide. The alkoxylation can be located both on the fatty-alcohol part and on the fatty-acid part, and also on both parts, of the ester oils. It is preferred according to the present invention, however, if the fatty alcohol was first alkoxylated and then was esterified with fatty acid. Formula (D4-II) depicts these bonds in generalized fashion.

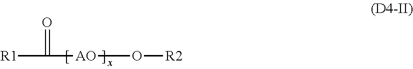

where
R1 denotes a saturated or unsaturated, branched or unbranched, cyclically saturated or cyclically unsaturated acyl residue having 6 to 30 carbon atoms,
AO denotes ethylene oxide, propylene oxide, or butylene oxide,
X denotes a number between 1 and 200, by preference 1 and 100, particularly preferably between 1 and 50, very particularly preferably between 1 and 20, highly preferably between 1 and 10 and most preferably between 1 and 5,
R2 denotes a saturated or unsaturated, branched or unbranched, cyclically saturated or cyclically unsaturated alkyl, alkenyl, alkinyl, phenyl, or benzyl residue having 6 to 30 carbon atoms. Examples of fatty-acid components used as residue R1 in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, as well as industrial mixtures thereof. Examples of the fatty-alcohol components as residue R2 in the ester oils are benzyl alcohol, isopropyl alcohol, capronyl alcohol, capryl alcohol, 2-ethylhexyl alcohol, caprinyl alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol, and brassidyl alcohol, as well as industrial mixtures thereof. An ester oil that is particularly preferred according to the present invention is obtainable, for example, under the INCI name PPG-3 Benzyl Ether Myristate, for example the commercial product Crodamol® STS.

Also to be understood as ester oils are:
dicarboxylic acid esters such as di-n-butyl adipate, di(2-ethylhexyl) adipate, di(2-ethylhexyl) succinate, and diisotridecyl acelaate, as well as diol esters such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate, and
symmetrical, asymmetrical, or cyclic esters of carbonic acid with fatty alcohols, for example glycerol carbonate or dicaprylyl carbonate (Cetiol® CC),
fatty acid triesters of saturated and/or unsaturated linear and/or branched fatty acids with glycerol,
fatty acid partial glycerides, i.e. monoglycerides, diglycerides, and industrial mixtures thereof. When industrial products are used, small quantities of triglycerides may still be present for manufacturing-related reasons. The partial glycerides preferably conform to formula (D4-I):

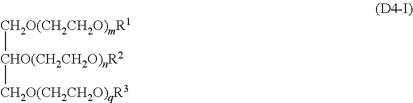

in which R¹, R² and R³, mutually independently, denote hydrogen or a linear or branched, saturated and/or unsaturated acyl residue having 6 to 22, preferably 12 to 18, carbon atoms, provided that at least one of these groups denotes an acyl residue and at least one of these groups denotes hydrogen. The sum (m+n+q) denotes 0 or numbers from 1 to 100, preferably 0 or 5 to 25. R1 preferably denotes an acyl residue and R² and R³ denote hydrogen, and the sum (m+n+q) is 0. Typical examples are mono- and/or diglycerides based on hexanoic acid, octanoic acid, 2-ethylhexanoic acid, decanoic acid, lauric acid, isotridecanoic acid, myristic acid, palmitic acid, palmoleic acid, stearic acid, isostearic acid, oleic acid, elaidic acid, petroselinic acid, linoleic acid, linolenic acid, elaeostearic acid, arachidic acid, gadoleic acid, behenic acid and erucic acid, as well as industrial mixtures thereof. Oleic acid monoglycerides are preferably used.

The ester oils are used in the agents according to the present invention in a quantity from 0.01 to 20 wt %, preferably 0.01 to 10.0 wt %, particularly preferably 0.01 to 7.5 wt %, highly preferably from 0.1 to 5.0 wt %. It is of course also possible according to the present invention to use more than two ester oils simultaneously. In every case the ester oils are selected so that the ester oils are combined with one another in accordance with their spreading values. It has been shown according to the present invention that it is particularly advantageous if a fast-spreading ester oil is combined with a slow-spreading ester oil. It is particularly advantageous if at least three and more ester oils are combined with one another. It is thereby possible for spreading, i.e. an easy and particularly effective ability to distribute the entire composition on the hair, to be implemented in outstanding fashion. Used for this purpose, in addition to a particularly slow-spreading ester oil and a particularly fast-spreading ester oil, are also at least one further, preferably multiple, ester oils that, in terms of their spreading behavior, fall between the slow-spreading and the fast-spreading ester oil.

The spreading behavior of ester oils can be very easily and simply determined by one skilled in the art. A first rough approximation is the molecular weight. To a first approximation, the spreading rate decreases with increasing molecular weight. These oil components influence, in particular, the smooth feel of the hair. Fast-spreading ester oils leave behind a definite smooth feel. Slow-spreading ester oils leave behind a distinctly long-lasting but not so pronounced smooth feel. It is not only the smooth feel that is influenced, however; spreading also influences the distribution of the entire composition on the hair. A uniform effect and distribution of the composition according to the present invention on the hair is achieved in particular by the use of at least two, particularly preferably three ester oils, of which in turn at least one must be an ester oil of a fatty acid with a fatty alcohol, particularly preferably, for example, isopropyl myristate, and another must be an ester of carbonic acid with fatty alcohol, preferably with dicaprylyl carbonate. When at least two ester oils are used, the ester oils are used in approximately equal-weight quantities. The use of somewhat higher proportions of the fast-spreading component is preferred. The preferred ratio is 1:1 to 2:1. If at least three ester oils are contained in the compositions according to the present invention, the third ester oil is then an ester oil of formula (D4-II). It is highly preferred in this case to use the ester oil having the designation PPG-3 Benzyl Ether Myristate.

At least one cationically charged polymeric compound is contained as a further particularly preferred ingredient. “Cationic polymers” are to be understood as polymers that comprise in the main chain and/or side chain a group that can be “temporarily” or “permanently” cationic.

Further cationic polymers according to the present invention are the so-called “temporarily cationic” polymers. These polymers usually contain an amino group that is present at certain pH values as a quaternary ammonium group and therefore cationically.

Both cationic and amphoteric or zwitterionic polymers can therefore be characterized by way of their cationic charge density. The polymers according to the present invention are notable for a charge density of at least 1 to 7 meq/g. A charge density of at least 2 to 7 meq/g is preferred in this context. A charge density equal to at least 3 meq/g to 7 meq/g is particularly preferred.

A further characteristic feature of the polymers according to the present invention is their molar weight. The “molar weight” of the particular polymer is understood as the molar weight indicated in the corresponding data sheets by the manufacturer according to its method. For selection of a suitable polymer, a molar weight of at least 50,000 g/u has proven suitable according to the present invention. Polymers having a molar weight of more than 100,000 g/u have proven particularly suitable. Polymers having a molar weight of more than 1,000,000 g/u are very particularly suitable.

Suitable polymers have, for the product of the cationic charge density and the molar weight, a value greater than 50,000. Polymers that have a value of at least 100,000 for this product are particularly suitable. Those polymers for which this product has a value greater than 150,000 are very particularly suitable. Most suitable are those polymers for which this product has a value of at least 1,000,000.

Those polymers that possess sufficient solubility in water or alcohol to go completely into solution in the agent according to the present invention are preferred.

The cationic polymers can be homo- or copolymers, the quaternary nitrogen groups being contained either in the polymer chain or, by preference, as a substituent on one or more of the monomers. The ammonium-group-containing monomers can be copolymerized with non-cationic monomers. Suitable cationic monomers are unsaturated, radically polymerizable compounds that carry at least one cationic group, in particular ammonium-substituted vinyl monomers such as, for example, trialkylmethacryloxyalkylammonium, trialkylacryloxyalkylammonium, dialkyldiallylammonium, and quaternary vinylammonium monomers having cyclic groups containing cationic nitrogens, such as pyridinium, imidazolium, or quaternary pyrrolidones, e.g. alkylvinylimidazolium, alkylvinylpyridinium, or alkyvinylpyrollidone salts. The alkyl groups of these monomers are by preference lower alkyl groups such as, for example, C1 to C7 alkyl groups, particularly preferably C1 to C3 alkyl groups.

The ammonium-group-containing monomers can be copolymerized with non-cationic monomers. Suitable comonomers are, for example, acrylamide, methacrylamide; alkyl and dialkylacrylamide, alkyl and dialkylmethacrylamide, alkyl acrylate, alkyl methacrylate, vinylcaprolactone, vinylcaprolactam, vinylpyrrolidone, vinyl esters, e.g. vinyl acetate, vinyl alcohol, propylene glycol, or ethylene glycol, the alkyl groups of these monomers being by preference C1 to C7 alkyl groups, particularly preferably C1 to C3 alkyl groups.

Suitable polymers having quaternary amine groups are, for example, the polymers described in the CTFA Cosmetic Ingredient Dictionary under the “Polyquaternium” designations, such as methylvinylimidazolium chloride/vinylpyrrolidone copolymer (Polyquaternium-16), or quaternized vinylpyrrolidone/dimethylaminoethyl methacrylate copolymer (Polyquaternium-11).

Suitable among the cationic polymers that can be contained in the agent according to the present invention is, for example, the vinylpyrrolidone/dimethylaminoethyl methacrylate methosulfate copolymer that is marketed under the commercial names Gafquat® 755 N and Gafquat® 734 by the GAF company, USA, and of which Gafquat® 734 is particularly suitable. Further cationic polymers are, for example, the copolymer of polyvinylpyrrolidone and imidazolimine methochloride marketed by BASF, Germany, under the trade name Luviquat® HM 550, the terpolymer of dimethyldiallylammonium chloride, sodium acrylate, and acrylamide marketed by the Calgon company, USA, under the trade name Merquat® Plus 3300, and the vinylpyrrolidone/methacrylamidopropyltrimethylammonium chloride copolymer marketed by the ISP company under the trade name Gafquat® HS 100.

Homopolymers of the general formula (P1),

{CH₂—[CR¹COO—(CH₂)ₘN⁺R²R³R⁴]}ₙX⁻ (P1)

in which
—R¹=—H or —CH₃,
R², R³ and R⁴ are selected, mutually independently, from C₁₋₄ alkyl, alkenyl, or hydroxyalkyl groups,
m=1, 2, 3 or 4,
n is a natural number, and
X⁻ is a physiologically acceptable organic or inorganic anion,
   as well as copolymers made up substantially of the monomer units presented in formula (Monomer-3), as well as nonionogenic monomer units, are particularly preferred cationic polymers. In the context of these polymers, those for which at least one of the following conditions apply are preferred according to the present invention:

R¹ denotes a methyl group

R², R³ and R⁴ denote methyl groups

m has the value of 2.

Possibilities as physiologically acceptable counter ions X⁻ are, for example, halide ions, sulfate ions, phosphate ions, methosulfate ions, and organic ions such as lactate, citrate, tartrate, and acetate ions. Halide ions, in particular chloride, are preferred.

A particularly suitable homopolymer is the poly(methacryloyloxyethyltrimethylammonium chloride) (crosslinked, if desired) having the INCI name Polyquaternium-37. Such products are available commercially, for example, under the designations Rheocare® CTH (Cosmetic Rheologies) and Synthalen® CR (3V Sigma). The crosslinking can be accomplished, if desired, with the aid of olefinically polyunsaturated compounds, for example divinylbenzene, tetraallyloxyethane, methylene bisacrylamide, diallyl ether, polyallylpolyglyceryl ether, or allyl ethers of sugars or sugar derivatives such as erythritol, pentaerythritol, arabitol, mannitol, sorbitol, sucrose, or glucose. Methylene bisacrylamide is a preferred cross-linking agent.

The homopolymer is preferably used in the form of a nonaqueous polymer dispersion that should comprise a polymer proportion not less than 30 wt %. Such polymer dispersions are obtainable commercially under the designations Salcare® SC 95 and Salcare® 96.

Copolymers having monomer units according to formula (Pmonomer-3) preferably contain acrylamide, methacrylamide, acrylic acid C₁₋₄ alkyl esters, and methacrylic acid C₁₋₄ alkyl esters as nonionogenic monomer units. Of these nonionogenic monomers, acrylamide is particularly preferred. These copolymers as well, as in the case of the homopolymers described above, can be crosslinked. A copolymer preferred according to the present invention is the crosslinked copolymer of acrylamide and methacryloyloxyethyltrimethylammonium chloride. Such copolymers, in which the monomers are present at a weight ratio of approximately 20:80, are commercially obtainable as an approx. 50% nonaqueous polymer dispersion under the designation Salcare® SC 92.

Suitable cationic polymers that are derived from natural polymers are cationic derivatives of polysaccharides, for example cationic derivatives of cellulose, starch, or guar. Chitosan and chitosan derivatives are also suitable. Cationic polysaccharides have the general formula (P-3)

G-O—B—N⁺RₐR_{b}R_{c}X⁻
G is an anhydroglucose residue, for example starch or cellulose anhydroglucose,
B is a divalent connecting group, for example alkylene, oxyalkylene, polyoxyalkylene, or hydroxyalkylene,
Rₐ, R_{b} and R_{c} are, mutually independently, alkyl, aryl, alkylaryl, arylalkyl, alkoxyalkyl, or alkoxyaryl each having up to 18 carbon atoms, the total number of carbon atoms in Rₐ, R_{b}, and R_{c} preferably being a maximum of 20,
X⁻ is a usual counter anion and is by preference chloride.

A cationic cellulose is marketed by Amerchol under the designation Polymer JR® 400 and has the INCI name Polyquaternium-10. A further cationic cellulose bears the INCI name Polyquaternium-24 and is marketed under the trade name Polymer LM-200 by Amerchol. Further commercial products are the compounds CelquatO H 100 and Celquat® L 200. The aforesaid commercial products are preferred cationic celluloses.

Suitable cationic guar derivatives are marketed under the commercial designation Jaguar® and have the INCI name Guar Hydroxypropyltrimonium Chloride. Particularly suitable cationic guar derivatives are additionally available commercially from the Hercules company under the designation N-Hance®. Further cationic guar derivatives are marketed by the Cognis company under the designation Cosmedia®. A preferred cationic guar derivative is the commercial product AquaCat® of the Hercules company. This raw material is a cationic guar derivative that is already predissolved.

A further particularly suitable natural cationic polymer is represented by hydrocolloids of the chitosan type. In contrast to most hydrocolloids, which are negatively charged in the biological pH range, chitosans represent cationic biopolymers under these conditions.

A suitable chitosan is marketed, for example, by the Kyowa Oil & Fat company, Japan, under the trade name Flonac®. A preferred chitosan salt is chitosonium pyrrolidonecarboxylate, which is marketed e.g. under the designation Kytamer® PC by the Amerchol company, USA. Appropriate chitosan derivatives are quaternized, alkylated, or hydroxyalkylated derivatives, for example hydroxyethyl- or hydroxybutylchitosan. Further chitosan derivatives are readily available commercially under the commercial designations Hydagen® CMF, Hydagen® HCMF, and Chitolam® NB/101.

Further preferred cationic polymers are, for example:
cationic alkyl polyglycosides,
cationized honey, for example the commercial product Honeyquat® 50,
polymeric dimethyldiallylammonium salts and copolymers thereof with esters and amides of acrylic acid and methacrylic acid. The products available commercially under the designations Merquat® 100 (poly(dimethyldiallylammonium chloride)) and Merquat® 550 (dimethyldiallylammonium chloride/acrylamide copolymer) are examples of such cationic polymers,
copolymers of vinylpyrrolidone with quaternized derivatives of dialkylaminoalkyl acrylate and methacrylate, such as, for example, vinylpyrrolidone/dimethylaminoethylmethacrylate copolymers quaternized with diethyl sulfate. Such compounds are obtainable commercially under the designations Gafquat® 734 and Gafquat® 755,
vinylpyffolidone/vinylimidazolium methochloride copolymers, such as those offered under the designations Luviquat® FC 370, FC 550, FC 905, and HM 552,
quaternized poly(vinylalcohol),
and the polymers known under the designations Polyquaternium-2, Polyquaternium-17, Polyquaternium-18, and Polyquaternium-27, having quaternary nitrogen atoms in the main polymer chain,
vinylpyrrolidone/vinylcaprolactam/acrylate terpolymers such as those having acrylic acid esters and acrylic acid amides as a third monomer module, and offered commercially e.g. under the designation Aquaflex® SF 40.

Also usable according to the present invention are the copolymers of vinylpyrrolidone such as those obtainable as the commercial products Copolymer 845 (manufacturer: ISP), Gaffix® VC 713 (manufacturer: ISP), Gafquat® ASCP 1011, Gafquat® HS 110, Luviquat® 8155, and Luviquat® MS 370.

Cationic polymers that are preferred according to the present invention are cationic cellulose derivatives and chitosan and its derivatives, in particular the commercial products Polymer® JR 400, Hydagen® HCMF, and Kytamer® PC, cationic guar derivatives, cationic honey derivatives, in particular the commercial product Honeyquat® 50, cationic alkyl polyglycosides according to German Patent 44 13 686, and polymers of the Polyquaternium-37 type.

Also to be included among the cationic polymers are cationized protein hydrolysates, in which context the underlying protein hydrolysate can derive from animals, for example from collagen, milk, or keratin, from plants, for example from wheat, corn, rice, potatoes, soy, or almonds, from marine life forms, for example from fish collagen or algae, or from biotechnologically obtained protein hydrolysates. Those cationic protein hydrolysates whose underlying protein component has a molecular weight from 100 to 25,000 dalton, preferably 250 to 5,000 dalton, are preferred. Also to be understood as cationic protein hydrolysates are quaternized amino acids and mixtures thereof. The cationic protein hydrolysates can furthermore also be further derivatized. Typical examples that may be mentioned of cationic protein hydrolysates and derivatives according to the present invention are the products listed under the INCI names in the “International Cosmetic Ingredient Dictionary and Handbook,” (seventh edition 1997, The Cosmetic, Toiletry, and Fragrance Association, 1101 17th Street, N.W., Suite 300, Washington, D.C. 20036-4702), and available commercially. The plant-based cationic protein hydrolysates and derivatives are very particularly preferred.

The cationic polymers are contained in the compositions according to the present invention preferably in quantities from 0.01 to 10 wt %, based on the entire agent. Quantities from 0.05 to 5 wt % are particularly preferred.

Amphoteric polymers are, like the cationic polymers, very particularly preferred polymers. The term “amphoteric polymers” encompasses both those polymers that contain in the molecule both free amino groups and free —COOH or —SO₃H groups and are capable of forming internal salts, and zwitterionic polymers, which contain quaternary ammonium groups and —COO⁻ or —SO₃⁻ groups in the molecule, and those polymers that contain —COOH or —SO₃H groups and quaternary ammonium groups.

Amphoteric and/or cationic polymers that are preferred according to the present invention are those polymerizates in which a cationic group derives from at least one of the following monomers:
monomers having quaternary ammonium groups of the general formula (Mono1)

R1-CH═CR²—CO—Z—(CₙH₂ₙ)—N⁽⁺⁾R³R⁴R⁵A⁽⁻⁾ (Mono1),
in which R¹ and R², mutually independently, denote hydrogen or a methyl group and R³, R⁴, and R⁵, mutually independently, denote alkyl groups having 1 to 4 carbon atoms, Z denotes an NH group or an oxygen atom, n is a whole number from 2 to 5, and A⁽⁻⁾ is the anion of an organic or inorganic acid, and
monomers having quaternary ammonium groups of the general formula (Mono2)

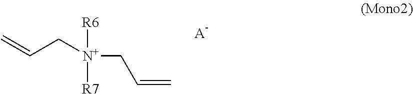

in which R⁶ and R⁷, mutually independently, denote a (C1 to C4) alkyl group, in particular a methyl group, and
A⁻ is the anion of an organic or inorganic acid.

If a cationic group of the amphoteric or cationic polymerizates derives from the monomer of formula (Mono1), the residues R³, R⁴, and R⁵ in formula (Mono1) preferably denote methyl groups, Z is preferably an NH group, and A⁽⁻⁾ preferably denotes a halide, methoxysulfate, or ethoxysulfate ion. In this case it is particularly preferred to use acrylamidopropyltrimethylammonium chloride as monomer (Mono1).

In formula (Mono2), A⁻ preferably denotes a halide ion, in particular chloride or bromide.

Preferred amphoteric polymers according to the present invention are polymers whose anionic group derives from at least one monomer of formula (Mono3):
monomeric carboxylic acids of the general formula (Mono3) or salts thereof with an organic or inorganic acid:

R⁸—CH═CR⁹COOH (Mono3)
in which R⁸ and R⁹, mutually independently, are hydrogen or methyl groups.

Acrylic acid is used as a monomer (Mono3) for the amphoteric polymerizates preferred according to the present invention.

Particularly preferred amphoteric polymers are copolymers of at least one monomer (Mono1) or (Mono2) with the monomer (Mono3), in particular copolymers of monomers (Mono2) and (Mono3). Amphoteric polymers used very particularly preferably according to the present invention are copolymerizates of diallyldimethylammonium chloride and acrylic acid. These copolymerizates are marketed under the INCI name Polyquaternium-22, inter alia with the commercial name Merquat® 280 (Nalco).

Furthermore, the amphoteric polymers according to the present invention can additionally contain, alongside a monomer (Mono1) or (Mono2) and a monomer (Mono3), a monomer (Mono4)
monomeric carboxylic acid amides of the general formula (Mono4)

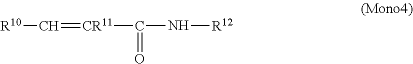

in which R¹⁰ and R¹¹, mutually independently, are hydrogen or methyl groups, and R¹² denotes a hydrogen atom or a (C₁ to C₄) alkyl group.

Amphoteric polymers that are based on a comonomer (Mono4) and are used very particularly preferably according to the present invention are terpolymers of diallyldimethylammonium chloride, acrylamide, and acrylic acid. These copolymerizates are marketed under the INCI name Polyquaternium-39, inter alia with the commercial name Merquat® Plus 3330 (Nalco).

Amphoteric polymers that are particularly preferred for use are those polymerizates that are made up substantially of

(i) monomers having quaternary ammonium groups of the general formula (Mono1)

R¹—CH═CR²—CO—Z—(CₙH₂ₙ)—N⁽⁺⁾R³R⁴R⁵A⁽⁻⁾ (Mono1)
in which R¹ and R², mutually independently, denote hydrogen or a methyl group and R³, R⁴, and R⁵, mutually independently, denote alkyl groups having 1 to 4 carbon atoms, Z denotes an NH group or an oxygen atom, n is a whole number from 2 to 5, and A⁽⁻⁾ is the anion of an organic or inorganic acid, and
   (ii) monomeric carboxylic acids of the general formula (Mono3)

R⁸—CH═CR⁹—COOH (Mono3)
in which R⁸ and R⁹, mutually independently, are hydrogen or methyl groups.

Those polymerizates in which the monomers used are of type (i) in which R³, R⁴, and R⁵ are methyl groups, Z is an NH group, and A⁽⁻⁾ is a halide, methoxysulfate, or ethoxysulfate ion, are very particularly preferred; acrylamidopropyltrimethylammonium chloride is a particularly preferred monomer (i). Acrylic acid is preferably utilized as monomer (ii) for the aforesaid polymerizates.

The amphoteric polymers can in general be used according to the present invention both directly and in a salt form that is obtained by neutralizing the polymerizates, for example with an alkali hydroxide.

The amphoteric polymers are contained in the agents according to the present invention preferably in quantities from 0.01 to 10 wt %, based on the entire agent. Quantities from 0.01 to 5 wt % are particularly preferred.

The cationic polymeric compounds preferred according to the present invention, as described above, are selected in particular from the cationic celluloses, the cationic guar derivatives, the cationic starches, and the Salcare grades and Merquat grades. The particularly preferred cationic polymeric compounds are Polyquatemium-37, Polyquaternium-80, Polyquaternium-22, Polyquaternium-10, Polyquaternium-11, and Polyquaternium-16.

In the highly preferred embodiments of the present invention, at least one of the aforementioned cationic polymeric compounds is used in each case as an ingredient. The embodiments are most highly preferred when Polyquaternium-37 or Polyquatemium-10, or both together, are used as a cationic polymer.

Contained as a further ingredient in the agents according to the present invention is at least one further quaternary imidazoline compound, i.e. a compound that comprises a positively charged imidazoline ring. The structure of these compounds also corresponds in principle to Formula Ia depicted above in the context of the description of the obligatory ingredient a), although it must be noted that the residues R have a chain length from 8 to 18 carbon atoms.

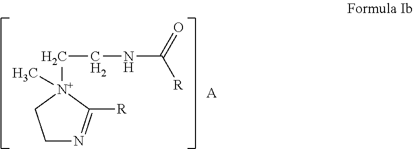

The residues R each denote, mutually independently, a saturated or unsaturated, linear or branched hydrocarbon residue having a chain length of 8 to 18 carbon atoms. The preferred compounds of formula Ib contain the same hydrocarbon residue for each residue R. The chain length of the residues R is preferably 12 to 18 carbon atoms. Compounds having a chain length of at least 16 carbon atoms, and very particularly having 18 carbon atoms, are particularly preferred. Preferred residues R are oleyl, palmityl, and stearyl. Counter ions according to the present invention are halides, for example fluoride, chloride, or bromide, alkyl sulfates such as methosulfate or ethosulfate, phosphates, citrate, tartrate, maleate, or fumarate. Examples that are particularly in accordance with the invention are obtainable, for example, under the INCI names Quaternium-27, Quaternium-72, and Quaternium-83.

The imidazolines are contained as ingredients in the compositions according to the present invention in quantities from 0.01 to 20 wt %, preferably in quantities from 0.05 to 10 wt %, and very particularly preferably in quantities from 0.1 to 7.5 wt %. The best results of all are obtained with quantities from 0.1 to 5 wt %, based in each case on the total composition of the respective agent.

Cationic surfactants of formula (Tkat-2) are used as very particularly preferred further cationic surfactants in addition to the combinations according to the present invention of cationic imidazolines a). These cationic surfactants are not only biodegradable but moreover are also particularly mild with regard to both the mucous membranes and, especially, the skin:

RCO—X—N⁺R¹R²R³R⁴A⁻ (Tkat-2),

in which
R denotes a substituted or unsubstituted, branched or straight-chain alkyl or alkenyl residue having 11 to 35 carbon atoms in the chain,
X denotes —O— or —NR5,
R¹ denotes an alkenyl group, having 2 to 6 carbon atoms, which can be unsubstituted or substituted, substitution with an —OH or —NH group being preferred in the case of a substitution,
R², R³ and R⁴ each denote, mutually independently, an alkyl or hydroxyalkyl group having 1 to 6 carbon atoms in the chain; the chain can be straight or branched. Examples of residues according to the present invention are methyl, ethyl, propyl, isopropyl, butyl, isobutyl, tert-butyl, pentyl, isopentyl, neopentyl, hexyl, isohexyl, hydroxyalkyl, dihydroxyalkyl, hydroxyethyl, hydroxypropyl, dihydroxypropyl, hydroxybutyl, dihydroxybutyl, trihydroxybutyl, trihydroxypropyl, dihydroxyethyl,
R⁵ denotes hydrogen or a C1 to C6 straight-chain or branched alkyl or alkenyl residue, which can also be substituted with a hydroxy group, in particular methyl, ethyl, propyl, isopropyl, butyl, isobutyl, tert-butyl, pentyl, isopentyl, neopentyl, hexyl, isohexyl, hydroxyethyl, hydroxypropyl, dihydroxypropyl, hydroxybutyl, dihydroxybutyl, trihydroxybutyl, trihydroxypropyl, dihydroxyethyl, and
A⁻ denotes a halide such as fluoride, chloride, or bromide, an alkyl sulfate such as a methosulfate or ethosulfate, a phosphate, a citrate, tartrate, maleate, or fumarate.

Within this structure class, compounds having one of the following structures are preferably used:

CH₃(CH₂)₂₀CONH(CH₂)₃—N⁺(CH₃)₂—CH₂CH₃A⁻ (Tkat-3)

CH₃(CH₂)₂₀CONH(CH₂)₃—N⁺(CH₃)₂—CH₂(CHOH)CH₂OH A⁻ (Tkat-4)

CH₃(CH₂)₂₀COOCH₂CHOHCH₂—N⁺(CH₃)₃A⁻ (Tkat-5)

CH₃(CH₂)₂₀CONH(CH₂)₃—N⁺(CH₃)₂—CH₂CH₂OHA⁻ (Tkat-6)

Examples of commercial products of this kind are Schercoquat BAS, Lexquat AMG-BEO, Akypoquat 131, or Incroquat Behenyl HE.

Even small quantities of the aforementioned cationic surfactants, in particular those that conform to formulas (Tkat-3) to (Tkat-6), are sufficient to distinctly improve the compositions according to the present invention, not only in terms of effects on the skin and hair. The entire composition is in fact thereby considerably improved, in particular in terms of its dermatological compatibility. Usual cosmetic compositions, for example mascara, eye shadow, lipstick, hair therapies, hair sprays, or leave-on products for application to the hair, can of course get into the eyes when used, and thereby considerably irritate the eyes. The compositions according to the present invention are notable here for much less irritation, even though they contain easily-spreading ester oils. It is in fact even possible, in particular, to award the designation “no more tears” to compositions according to the present invention.

The cationic surfactants of formula (Tkat-2) are contained in the compositions according to the present invention in quantities from 0.01 to 20 wt %, preferably in quantities from 0.01 to 10 wt %, and very particularly preferably in quantities from 0.1 to 7.5 wt %. The best results of all are obtained in this context with quantities from 0.1 to 5 wt %, based in each case on the total composition of the respective agent.

In addition, in an embodiment, esterquats in accordance with formula (Tkat1-2) can be used:

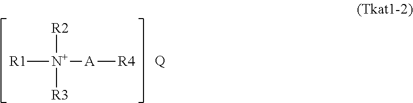

in which the residues R1, R2, and R3 are each mutually independent and can be the same or different. Residues R1, R2, and R3 denote:
a branched or unbranched alkyl residue having 1 to 4 carbon atoms, which can contain at least one hydroxyl group, for example methyl, ethyl, propyl, isopropyl, butyl, isobutyl, tert-butyl, hydroxyethyl, hydroxymethyl, or

a saturated or unsaturated, branched or unbranched, or a cyclic unsaturated or unsaturated alkyl residue having 6 to 30 carbon atoms, which can contain at least one hydroxyl group, or
an aryl or alkaryl residue, for example phenyl or benzyl,
the residue (-A-R4), provided that at most two of the residues R1, R2, or R3 can denote this residue:
   The residue -(A-R4) is contained at least 1 to 3 times.

In this, A denotes:
1) —(CH₂)ₙ—, where n=1 to 20, by preference n=1 to 10, and particularly preferably n=1 to 5, or
2) —(CH₂—CHR5—O)ₙ—, where n=1 to 200, by preference 1 to 100, particularly preferably 1 to 50, and particularly preferably 1 to 20 where R5 has the meaning of hydrogen, methyl, or ethyl,
   and R4 denotes:
1) R6—O—CO—, in which R6 is a saturated or unsaturated, branched or unbranched, or a cyclic saturated or unsaturated alkyl residue having 6 to 30 carbon atoms, which can contain at least one hydroxy group, and which if applicable can be further oxyethylated with 1 to 100 ethylene oxide units and/or 1 to 100 propylene oxide units, or
2) R7—CO—, in which R7 is a saturated or unsaturated, branched or unbranched, or a cyclic saturated or unsaturated alkyl residue having 6 to 30 carbon atoms, which can contain at least one hydroxy group, and which if applicable can be further oxyethylated with 1 to 100 ethylene oxide units and/or 1 to 100 propylene oxide units,
   and Q denotes a physiologically acceptable organic or inorganic anion that by preference is selected from the halides, for example fluoride, chloride, bromide, the sulfates, for example the methosulfates of the general formula RSO3— in which R has the meaning of saturated or unsaturated alkyl residues having 1 to 4 carbon atoms, the phosphates, or anionic residues of organic acids such as maleate, fumarate, oxalate, tartrate, citrate, lactate, or acetate.

Esterquats are known substances that contain both at least one ester function and at least one quaternary ammonium group as a structural element. Preferred esterquats are quaternized ester salts of fatty acids with triethanolamine, quaternized ester salts of fatty acids with diethanolalkylamines, and quaternized ester salts of fatty acids with 1,2-dihydroxypropyldialkylamines. Such products are marketed, for example, under the trademarks Rewoquat®, Stepantex®, Dehyquart®, and Armocare®. Examples of such esterquats are the products Armocare® VGH-70—and N,N-bis(2-palmitoyloxyethyl)dimethylammonium chloride—as well as Dehyquart® F-75, Dehyquart® C-4046, Dehyquart® L-80, Dehyquart® F-30, Dehyquart® AU-35, Rewoquat® WE18, Rewoquat® WE38 DPG, and Stepantex® GS 90.

Further compounds of formula (Tkat1-2) that are particularly preferred according to the present invention belong to formula (Tkat1-2.1), the cationic betaine esters:

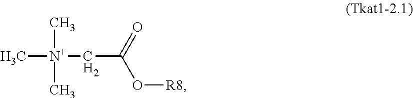

in which R8 corresponds in meaning to R7.

The esterquats are contained in the compositions according to the present invention in quantities from 0.01 to 20 wt %, preferably in quantities from 0.01 to 10 wt %, and very particularly preferably in quantities from 0.1 to 7.5 wt %. The best results of all are obtained in this context with quantities from 0.1 to 5 wt %, based in each case on the total composition of the respective agent.

In an embodiment, monoalkyltrimethylammonium salts having an alkyl residue chain length from 16 to 24 carbon atoms can be contained as a further ingredient.

These compounds have the structure depicted in formula (Tkat1-1):

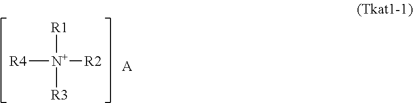

in which R1, R2, and R3 each denote a methyl group and R4 denotes a saturated, branched or unbranched alkyl residue having a chain length from 16 to 24 carbon atoms, and A denotes an ion selected from the physiologically acceptable anions. Examples of the anion that may be recited are the halides, fluoride, chloride, bromide, sulfate (methosulfate) of the general formula RSO₃⁻ in which R has the meaning of a saturated or unsaturated alkyl residue having 1 to 4 carbon atoms, or anionic residues of organic acids such as maleate, fumarate, oxalate, tartrate, citrate, lactate, or acetate.

Particularly preferred compounds of formula (Tkat1-1) have chloride or methosulfate as an anion, with a methyl group as residue R and furthermore, as residue R4, a saturated, branched or unbranched alkyl residue, very particularly preferably an unbranched alkyl residue having a chain length from 18 to 24, highly preferably having a chain length from 22 to 24 carbon atoms.

Examples of compounds of formula (Tkat1-1) are cetyltrimethylammonium chloride, cetyltrimethylammonium bromide, cetyltrimethylammonium methosulfate, stearyltrimethylammonium chloride, behenyltrimethylammonium chloride, behenyltrimethylammonium bromide, and behenyltrimethylammonium methosulfate. Cetyltrimethylammonium salts and behenyltrimethylammonium salts are preferred. The latter are particularly preferred in the form of the methosulfates and bromides. Cetyltrimethylammonium methosulfate and behenyltrimethylammonium methosulfate are the most preferred, and behenyltrimethylammonium methosulfate is highly preferred.

The compounds of formula (Tkat1-1) are used in the compositions according to the present invention in a quantity from 0.01 to 5.0 wt %. Preferably, 0.1 to 5.0 wt % is used. Quantities from 0.1 to 3.0 wt % are particularly preferred. The quantitative indications refer in each case to the entire composition.

In a particularly preferred embodiment of the invention, the agents according to the present invention further contain at least one amine and/or cationized amine, in particular an amidoamine and/or cationized amidoamine, having the following structural formulas:

R¹—NH—(CH₂)ₙ—NR²R³ (Tkat7) and/or

R¹—NH—(CH₂)ₙ—NR²R³R⁴ (Tkat8)

in which
R¹ denotes an acyl or alkyl residue having 6 to 30 carbon atoms, which can be branched or unbranched, saturated or unsaturated, and such that the acyl residue and/or alkyl residue can contain at least one OH group, and
R², R³, and R⁴ each denote, mutually independently, hydrogen or an alkyl residue having 1 to 4 carbon atoms, which can be the same or different, saturated or unsaturated, and
X-denotes an anion, and
n denotes a whole number between 1 and 10.

The anion is selected from the physiologically acceptable anions. Examples thereof that may be recited are the halide ions, fluoride, chloride, bromide, sulfate of the general formula RSO₃⁻ in which R has the meaning of a saturated or unsaturated alkyl residue having 1 to 4 carbon atoms, or anionic residues of organic acids such as maleate, fumarate, oxalate, tartrate, citrate, lactate, or acetate.

A composition in which the amine and/or quaternized amine in accordance with the general formulas (Tkat7) and/or (Tkat8) is an amidoamine and/or a quaternized amidoamine, in which R¹ denotes a branched or unbranched, saturated or unsaturated acyl residue having 6 to 30 carbon atoms, which can contain at least one OH group, is preferred. A fatty acid residue made up of oils and waxes, in particular natural oils and waxes, is preferred in this context. Lanolin, beeswax, or candelilla wax are appropriate examples thereof.

The alkylamidoamines are usually produced by amidation of natural or synthetic fatty acids and fatty acid cuts with dialkylaminoamines.

Also preferred are those amidoamines ad/or quaternized amidoamines in which R², R³, and/or R⁴ in formulas (Tkat7) and/or (Tkat8) denote a residue in accordance with the general formula CH2CH2OR5 in which R⁵ can have the meaning of alkyl residues having 1 to 4 carbon atoms, hydroxyethyl, or hydrogen. The preferred value of n in the general formulas (Tkat7) and/or (Tkat8) is a whole number between 2 and 5.

Also preferred are amidoamines and/or quaternized amidoamines of the general formulas (Tkat7) and/or (Tkat8) in which the anion X⁻ is a halide ion or a compound of the general formula RSO₃⁻, in which R has the meaning of a saturated or unsaturated alkyl residue having 1 to 4 carbon atoms.

The alkyl residue having 1 to 4 carbon atoms of R², R³, and R⁴, and/or the alkyl residue having 1 to 4 carbon atoms of RSO₃⁻ in the general formula (Tkat7) and/or (Tkat8), can contain at least one hydroxyl group.

The alkylamidoamines both can be present as such, and can be converted by protonation in a corresponding acid solution into a quaternary compound in the composition. The cationic alkylamidoamines are preferred according to the present invention.

The following, for example, are appropriate as amidoamines to be used according to the present invention, which if applicable can be quaternized: Witcamine 100 (Witco, INCI name: Cocamidopropyl Dimethylamine), Incromine BB (Croda, INCI name: Behenamidopropyl Dimethylamine), Mackine 401 (McIntyre, INCI name: Isostearylamidopropyl Dimethylamine) and other Mackine grades, Adogen S18V (Witco, INCI name: Stearylamidopropyl Dimethylamine) and, as permanently cationic aminoamines: Rewoquat RTM 50 (Witco Surfactants GmbH, INCI name: Ricinoleamidopropyltrimonium Methosulfate), Empigen CSC (Albright & Wilson, INCI name: Cocamidopropyltrimonium Chloride), Swanol Lanoquat DES-50 (Nikko, INCI name: Quatemium-33), Rewoquat UTM 50 (Witco Surfactants GmbH, Undecyleneamidopropyltrimonium Methosulfate).

The amidoamines or quaternized amidoamines in accordance with the general formulas (Tkat7) and (Tkat8) can be used individually or in any combinations with one another, being contained in quantities between 0.01 and 20 wt %, preferably in quantities from 0.01 to 10 wt %, and very particularly preferably in quantities from 0.1 to 7.5 wt %. The best results of all are obtained in this context with quantities from 0.1 to 5 wt %, based in each case on the entire composition of the respective agent.

Cosmetic oils can furthermore be used additionally with the active-substance combination (A) according to the present invention. These oily substances preferably have a melting point lower than 50° C., particularly preferably lower than 45° C., very particularly preferably lower than 40° C., highly preferably lower than 35° C., and most preferably the cosmetic oils are flowable at a temperature below 30° C. These oils are defined and described in more detail below:

Included among the natural and synthetic cosmetic oils are, for example:
Vegetable oils. Examples of such oils are sunflower oil, olive oil, soybean oil, rapeseed oil, almond oil, jojoba oil, orange oil, wheat germ oil, peach-kernel oil, and the liquid components of coconut oil. Also suitable, however, are other triglyceride oils such as the liquid components of beef tallow, as well as synthetic triglyceride oils.
Liquid paraffin oils, isoparaffin oils, and synthetic hydrocarbons, as well as di-n-alkyl ethers having a total of between 12 and 36 carbon atoms, in particular 12 to 24 carbon atoms, such as, for example, di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether, and n-hexyl-n-undecyl ether, as well as ditert-butyl ether, diisopentyl ether, di-3-ethyldecyl ether, tert-butyl-n-octyl ether, isopentyl-n-octyl ether, and 2-methylpentyl-n-octyl ether. The compounds 1,3-di-(2-ethylhexyl)cyclohexane (Cetiol® S) and di-n-octyl ether (Cetiol® OE), available as commercial products, can be preferred.

Suitable natural oils are, for example, amaranth seed oil, apricot kernel oil, argan oil, avocado oil, babassu oil, cottonseed oil, borage seed oil, camelina oil, thistle oil, peanut oil, pomegranate oil, grapefruit seed oil, hemp oil, hazelnut oil, elderberry seed oil, blackcurrant seed oil, jojoba oil, cocoa butter, linseed oil, macadamia nut oil, corn oil, almond oil, marula oil, evening primrose oil, olive oil, palm oil, rapeseed oil, rice oil, sea buckthorn fruit oil, sesame oil, shea butter, soybean oil, sunflower oil, grapeseed oil, walnut oil, or wild rose oil. Preferred oils are argan oil, amaranth seed oil, and shea butter.

The quantity of natural and synthetic oily substances to be used in the agents utilized according to the present invention is usually 0.1 to 30 wt % based on the entire agent, preferably 0.1 to 20 wt % and in particular 0.1 to 15 wt %.

In many cases the agents contain at least one surface-active substance, both anionic and zwitterionic, ampholytic, nonionic, and cationic surface-active substances being suitable in principle. Selection of the surface-active substances is based on the nature of the agent. In the case of a shampoo, in particular at least one surfactant from the group of the anionic, zwitterionic, or nonionic surface-active substances is selected. It is preferred in this context that at least one anionic and at least one zwitterionic surface-active substance be selected. Particularly preferably in this context, these surface-active substances are selected from the group of the particularly mild surface-active substances. In many cases, however, it has proven advantageous to select the surfactants from anionic, zwitterionic, or nonionic surfactants. The ratio between anionic and zwitterionic surface-active substances is in this context between 10:1 and 1:5, a ratio of 5:1 to 1:2 being particularly preferred.

If the agent represents a hair-conditioning composition, then cationic and/or nonionic surface-active substances are preferably selected as surface-active substances. Here again, in turn, selection of the so-called mild surface-active substances is particularly preferred.

All anionic surface-active substances suitable for use on the human body are suitable as anionic surfactants (Tanion) in preparations according to the present invention. Typical examples of anionic surfactants are:
linear and branched fatty acids having 8 to 30 carbon atoms (soaps),
ethercarboxylic acids of the formula R—O—(CH₂—CH₂O)ₓ—CH₂—COOH, in which R is a linear alkyl group having 8 to 30 carbon atoms and x=0 or is 1 to 16,
acyl isethionates having 8 to 24 carbon atoms in the acyl group. If fatty acids having 8 to 24 carbon atoms are used for esterification, i.e. for example lauric, myristic, palmitic, or stearic acid, or even industrial fatty acid fractions such as the C12 to C18 fatty acid fraction obtainable from coconut fatty acid, then the C12 to C18 acyl isethionates preferably suitable according to the present invention are obtained,
sulfosuccinic acid mono- and -diallyl esters having 8 to 24 carbon atoms in the alkyl group, and sulfosuccinic acid monoalkylpolyoxyethyl esters having 8 to 24 carbon atoms in the alkyl group and 1 to 6 oxyethyl groups,
linear alkanesulfonates having 8 to 24 carbon atoms,
linear alpha-olefinsulfonates having 8 to 24 carbon atoms,
alpha-sulfofatty acid methyl esters of fatty acids having 8 to 30 carbon atoms,
alkyl sulfates and alkylpolyglycol ether sulfates of the formula R—O(CH₂—CH₂O)ₓ—OSO₃H, in which R is a preferably linear alkyl group having 8 to 30 carbon atoms and x=0 or is 1 to 12,
hydroxysulfonates substantially corresponding to at least one of the two following formulas, or mixtures thereof, as well as salts thereof: CH₃—(CH₂)_{y}—CHOH—(CH₂)ₚ—(CH—SO₃M)-(CH₂)_{z}—CH₂—O—(CₙH₂ₙO)ₓ—H and/or CH₃—(CH₂)_{y}—CH₃—(CH₂)_{y}—(CH—SO₃M)-(CH₂)ₚ—CHOH—(CH₂)_{z}—CH₂—O—(CₙH₂ₙO)ₓ—H, such that in both formulas y and z=0 or whole numbers from 1 to 18, p=0, 1, or 2, and the sum (y+z+p) is a number from 12 to 18, x=0 or a number from 1 to 30, and n is a whole number from 2 to 4,
sulfated hydroxyalkylpolyethylene glycol ethers and/or hydroxyalkylenepropylene glycol ethers of the formula R¹—(CHOSO₃M)-CHR³—(OCHR⁴—CH₂)—OR², where R1 denotes a linear alkyl residue having 1 to 24 carbon atoms, R² a linear or branched, saturated alkyl residue having 1 to 24 carbon atoms, R³ denotes hydrogen or a linear alkyl residue having 1 to 24 carbon atoms, R⁴ denotes hydrogen or a methyl residue, and n a number in the range from 0 to 12, and furthermore the total number of carbon atoms contained in R1 and R3 is 2 to 44,
sulfonates of unsaturated fatty acids having 8 to 24 carbon atoms and 1 to 6 double bonds,
esters of tartaric acid and citric acid with alcohols that represent addition products of approximately 2 to 15 molecules of ethylene oxide and/or propylene oxide with fatty alcohols having 8 to 22 carbon atoms.
alkyl and/or alkenyl ether phosphates of the formula

R¹(OCH₂CH₂)ₙ—O(PO—OX)—OR²
in which R¹ preferably denotes an aliphatic hydrocarbon residue having 8 to 30 carbon atoms, R² denotes hydrogen, a (CH₂CH₂O)ₙR² residue, or X, n denotes numbers from 1 to 10
sulfated fatty acid alkylene glycol esters of the formula RCO(AlkO)ₙSO₃M, in which RCO— denotes a linear or branched, aliphatic, saturated and/or unsaturated acyl residue having 6 to 22 carbon atoms, Alk denotes CH₂CH₂, CHCH₃CH₂, and/or CH₂CHCH₃, n denotes numbers from 0.5 to 5,
monoglyceride sulfates and monoglyceride ether sulfates of the formula R⁸OC—(OCH₂CH₂)ₓ—OCH₂—[CHO(CH₂CH₂O)_{y}H]—CH₂O(CH₂CH₂O)_{z}—SO₃X,
in which R⁸CO denotes a linear or branched acyl residue having 6 to 22 carbon atoms, x, y, and z in total denote 0 or numbers from 1 to 30, preferably 2 to 10. Typical examples of monoglyceride (ether) sulfates suitable for purposes of the invention are the reaction products of lauric acid monoglyceride, coconut fatty acid monoglyceride, palmitic acid monoglyceride, stearic acid monoglyceride, oleic acid monoglyceride, and tallow fatty acid monoglyceride, and their ethylene oxide adducts with sulfur trioxide or chlorosulfonic acid in the form of their sodium salts. It is preferable to use monoglyceride sulfates in which R⁸CO denotes a linear acyl residue having 8 to 18 carbon atoms,
amide ethercarboxylic acids, R¹—CO—NR²—CH₂CH₂—O—(CH₂CH₂O)ₙCH₂COOM, where R¹ is a straight-chain or branched alkyl or alkenyl residue having a number of carbon atoms in the chain from 2 to 30, n denotes a whole number from 1 to 20, and R² denotes hydrogen, a methyl, ethyl, propyl, isopropyl, n-butyl, tert-butyl, or isobutyl residue. Products of this kind are obtainable, for example, from the Chem-Y company under the product designation Akypo®.
acyl glutamates of the formula XOOC—CH₂CH₂CH(C(NH)OR)—COOX, in which RCO denotes a linear or branched acyl residue having 6 to 22 carbon atoms and 0 and/or 1, 2, or 3 double bonds,
condensation products of a water-soluble salt of a water-soluble protein hydrolysate with a C8 to C30 fatty acid. Such products are obtainable under the trade names Lamepon®, Maypon®, Gluadin®, Hostapon® KCG, or Amisoft®,
acyl lactylates,
hydroxy mixed ether sulfates,
acyl sarcosides having 8 to 24 carbon atoms in the acyl group,
acyl taurides having 8 to 24 carbon atoms in the acyl group,
acyl isethionates having 8 to 24 carbon atoms in the acyl group,
sulfosuccinic acid mono- and dialkyl esters,
alkylpolyglycol ether sulfates of the formula R—O(CH₂—CH₂O)ₓ—OSO₃H,
esters of tartaric acid and citric acid with alcohols that represent addition products of approximately 2 to 15 molecules of ethylene oxide and/or propylene oxide with fatty alcohols having 8 to 22 carbon atoms,
alkyl and/or alkenyl ether phosphates of the formula R¹(OCH₂CH₂)ₙ—O—(PO—OX)—OR²,
monoglyceride sulfates and monoglyceride ether sulfates of the formula ROC—(OCH₂CH₂)_{X}—OCH₂—[CHO(CH₂CH₂O)_{y}H]-13 CH₂O(CH₂CH₂O)_{Z}—SO₃X,
acyl glutamates, and
acyl aspartates.

If the mild anionic surfactants contain polyglycol ether chains, it is very particularly preferred that they exhibit a restricted homolog distribution. It is further preferred in the case of mild anionic surfactants having polyglycol ether units that the number of glycol ether groups be equal to 1 to 20, preferably 2 to 15, particularly preferably 2 to 12. Particularly mild anionic surfactants having polyglycol ether groups without a restricted homolog distribution can also be obtained, for example if the one hand the number of polyglycol ether groups is equal to 4 to 12, and Zn or Mg ions are selected as a counter ion. One example of this is the commercial product Texapon® ASV.

All the mild anionic surfactants recited hitherto and hereinafter can of course also be used in the form of their salts. Particularly suitable mild anionic surfactants are present respectively in the form of the lithium, magnesium, zinc, sodium, potassium, and ammonium as well as mono-, di-, and trialkanolammonium salts having 1 to 4 carbon atoms in the alkanol group. The preferred ammonium ions are, in addition to the ammonium ion as such, monomethylammonium, dimethylammonium, trimethylammonium, monoethylammonium, diethylammonium, triethylammonium, monopropylammonium, dipropylammonium, tripropylammonium, monoisopropylammonium, diisopropylammonium, triisopropylammonium, monobutylammonium, dibutylammonium, tributylammonium, monoisobutylammonium, diisobutylammonium, triisobutylammonium, mono-t-butylammonium, di-t-butyl-ammonium, tri-t-butylammonium ions, as well as mixed ammonium ions such as, for example, methylethylammonium, dimethylethylammonium, methyldiethylammonium, methylpropylammonium, methylethylpropylammonium, ethyldiisopropylammonium, ethyldibutylammonium, ethyldiisobutylammonium ions, etc. The teaching of the present invention of course also encompasses the further ammonium ions, not explicitly recited, of these alkanol ammonium salts.

Further mild anionic surfactants that are used in particularly preferred fashion in the composition according to the present invention are alkyl and/or alkenyl oligoglycoside carboxylates, sulfates, phosphates, and/or isethionates that are derived from alkyl and/or alkenyl oligoglycosides.

“Zwitterionic surfactants” (Tzwitter) refers to those surface-active compounds that contain in the molecule at least one quaternary ammonium group and at least one —COO⁽⁻⁾ or SO3⁽⁻⁾ group. Particularly suitable zwitterionic surfactants are the so-called betaines, such as the N-alkyl-N,N-dimethylammonium glycinates, for example cocalkyldimethylammonium glycinate, N-acylaminopropyl-N,N-dimethylammonium glycinates, for example cocacylaminopropyldimethylammonium glycinate, and 2-alkyl-3-carboxymethyl-3-hydroxyethylimidazolines, having in each case 8 to 18 carbon atoms in the alkyl or acyl group, as well as cocacylaminoethylhydroxyethylcarboxymethyl glycinate. A preferred zwitterionic surfactant is the fatty acid amide derivative known by the INCI name Cocamidopropyl Betaine.

Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids, having in each case approximately 8 to 24 carbon atoms in the alkyl group. Typical examples of amphoteric or zwitterionic surfactants are alkyl betaines, alkylamidobetaines, aminopropionates, aminoglycinates, imidazolinium betaines, and sulfobetaines.

Particularly preferred ampholytic surfactants are N-cocalkylaminopropionate, cocacylaminoethylaminopropionate, and C12 to C18 acyl sarcosine.

Nonionic surfactants (Tnio) contain as a hydrophilic group, for example, a polyol group, a polyalkylene glycol ether group, or a combination of a polyol and polyglycol ether group. Such compounds are, for example:
addition products of 2 to 50 mol ethylene oxide and/or 0 to 5 mol propylene oxide with linear and branched fatty alcohols having 6 to 30 carbon atoms, the fatty alcohol polyglycol ethers or fatty alcohol polypropylene glycol ethers, or mixed fatty alcohol polyethers,
addition products of 2 to 50 mol ethylene oxide and/or 0 to 5 mol propylene oxide with linear and branched fatty acids having 6 to 30 carbon atoms, the fatty acid polyglycol ethers or fatty acid polypropylene glycol ethers or mixed fatty acid polyethers,
addition products of 2 to 50 mol ethylene oxide and/or 0 to 5 mol propylene oxide with linear and branched alkylphenols having 8 to 15 carbon atoms in the alkyl group, the alkylphenol polyglycol ethers or alkylphenol polypropylene glycol ethers or mixed alkylphenol polyethers,
addition products, end-capped with a methyl or C2 to C6 alkyl group, of 2 to 50 mol ethylene oxide and/or 0 to 5 mol propylene oxide with linear and branched fatty alcohols having 8 to 30 carbon atoms, with fatty acids having 8 to 30 carbon atoms, and with alkylphenols having 8 to 15 carbon atoms in the alkyl group, such as, for example, the grades obtainable under the marketing designations Dehydrol® LS, Dehydrol® LT (Cognis),
C12 to C30 fatty acid mono- and diesters of addition products of 1 to 30 mol ethylene oxide with glycerol,
addition products of 5 to 60 mol ethylene oxide with castor oil and hardened castor oil,
polyol fatty acid esters such as, for example, the commercial product Hydagen® HSP (Cognis), or Sovermol® grades (Cognis),
alkoxylated triglycerides,
alkoxylated fatty acid alkyl esters of formula (Tnio-I):

R¹CO—(OCH₂CHR²)_{w}OR² (Tnio-I),
in which R¹CO denotes a linear or branched, saturated and/or unsaturated acyl residue having 6 to 22 carbon atoms, R² denotes hydrogen or methyl, R³ denotes linear or branched alkyl residues having 1 to 4 carbon atoms, and w denotes numbers from 1 to 20,
amine oxides,
hydroxy mixed ethers of the formula R¹O[CH₂CH(CH₃)O]ₓ(CH₂CHR²O)_{y}[CH₂CH(OH)R³]_{z} where R¹ denotes a linear or branched, saturated or unsaturated alkyl and/or alkenyl residue having 2 to 30 carbon atoms, R² denotes hydrogen, a methyl, ethyl, propyl, or isopropyl residue, R³ denotes a linear or branched alkyl residue having 2 to 30 carbon atoms, x denotes 0 or a number from 1 to 20, Y denotes a number from 1 to 30, and z denotes the number 1, 2, 3, 4, or 5.
sorbitan fatty acid esters and addition products of ethylene oxide with sorbitan fatty acid esters, for example the polysorbates,

sugar fatty acid esters and addition products of ethylene oxide with sugar fatty acid esters,
addition products of ethylene oxide with fatty acid alkanolamides and fatty amines,
sugar surfactants of the alkyl and alkenyl oligoglycoside types, according to formula (E4-II)

R⁴O—[G]ₚ (Tnio-2)
in which R⁴ denotes an alkyl or alkenyl residue having 4 to 22 carbon atoms, G denotes a sugar residue having 5 or 6 carbon atoms, and p denotes numbers from 1 to 10.
sugar surfactants of the fatty acid N-alkylpolyhydroxyalkylamide types, a nonionic surfactant of formula (Tnio-3)

R⁵O—NR⁶—[Z] (Tnio-3)
in which R⁵CO denotes an aliphatic acyl residue having 6 to 22 carbon atoms, R⁶ denotes hydrogen, an alkyl or hydroxyalkyl residue having 1 to 4 carbon atoms, and [Z] denotes a linear or branched polyhydroxyalkyl residue having 3 to 12 carbon atoms and 3 to 10 hydroxyl groups.

The sugar surfactants can be contained in the agents used according to the present invention preferably in quantities from 0.1 to 20 wt %, based on the entire agent. Quantities from 0.5 to 15 wt % are preferred, and quantities from 0.5 to 7.5 wt % are very particularly preferred.

Further typical examples of nonionic surfactants are fatty acid amide polyglycol ethers, fatty amine polyglycol ethers, mixed ethers or mixed formals, protein hydrolysates (especially wheat-based vegetable products), and polysorbates.

The alkylene oxide addition products with saturated linear fatty alcohols and fatty acids, having respectively 2 to 30 mol ethylene oxide per mol fatty alcohol or fatty acid, and the sugar surfactants, have proven to be preferred nonionic surfactants. Preparations having outstanding properties are likewise obtained if they contain, as nonionic surfactants, fatty acid esters of ethoxylated glycerol.

These compounds are characterized by the following parameters: The alkyl residue R contains 6 to 22 carbon atoms and can be both linear and branched. Primary linear aliphatic residues, and aliphatic residues methyl-branched in the 2-position, are preferred. Such alkyl residues are, for example, 1-octyl, 1-decyl, 1-lauryl, 1-myristyl, 1-cetyl, and 1-stearyl. 1-Octyl, 1-decyl, 1-lauryl, and 1-myristyl are particularly preferred. When so-called “oxo alcohols” are used as the initial materials, compounds having an odd number of carbon atoms in the alkyl chain predominate.

The compounds having alkyl groups used as surfactants can in each case be uniform substances. It is preferred as a rule, however, to proceed from natural vegetable or animal raw materials when producing these substances, so that substance mixtures having different alkyl chain lengths, dependent on the particular material, are obtained.

A very particularly preferred embodiment additionally contains at least one further cationic compound alongside the obligatory ingredients in accordance with the statements made above. This cationic compound can be a further cationic surfactant and/or a further cationic polymer or at least one cationic surfactant and one cationic polymer, respectively. “Cationic” is also to be understood, for purposes of the invention under this embodiment, as an amphoteric polymer. A description and definition of both the further cationic surfactants and the cationic and/or amphoteric polymers is provided later in the specification at the location indicated.

Cationic surfactants (Tkat) are generally derived from ammonium ions and possess a

(NR¹R²R³R⁴)⁺A⁻ (Tkat1)

structure with a correspondingly negatively charged counter ion A. Cationic ammonium compounds of this kind are very familiar to one skilled in the art. The residues R¹ to R⁴ can each denote, mutually independently, straight-chain, branched, cyclic, aromatic, saturated, or unsaturated alkyl and/or alkenyl residues having at least 1 to 30 carbon atoms, hydrogen, —OFT, or hydroxyethyl.

Further cationic surfactants are, for example, the esterquats or the imidazolium compounds. Particularly preferred for use according to the present invention are cationic surfactants (Tkat) of the quaternary ammonium compound type, the esterquat type, and the imidazoline type. These have already been extensively described above.

Preferred quaternary ammonium compounds are ammonium halides, in particular chlorides and bromides, such as dialkyldimethylammonium chlorides, e.g. distearyldimethylammonium chloride, lauryldimethylammonium chloride, lauryldimethylbenzylammonium chloride, and tricetylmethylammonium chloride.

Cationic compounds having at least two behenyl residues can be used with particular preference. These substances are commercially obtainable, for example, under the designations Genamin® KDMP (Clariant).

A further example of a quaternary sugar derivative usable as a cationic surfactant is represented by the commercial product Glucquat® 100, according to INCI nomenclature a “Lauryl Methyl Gluceth-10 Hydroxypropyl Dimonium Chloride.”

The teaching of the present invention of course also encompasses the realization that mixtures of at least two cationic surfactants can be used. In this case the cationic surfactants are preferably selected from at least two different structure classes of cationic surfactants.

These further optional cationic surfactants (Tkat) are contained in the agents used according to the present invention preferably in quantities from 0.05 to 10 wt %, based on the entire agent. Quantities from 0.1 to 5 wt % are particularly preferred.

In the case of the cationic surfactants as well, it is of course also in accordance with the present invention to preferentially select mild cationic surfactants. Included among the corresponding mild cationic surfactants are, in particular, alkylamidoamines, quaternized amidoamines, esterquats, cationic surfactants of formula (Tkat-2), and cationic surfactants having at least one behenyl residue in the molecule.

Cationic, zwitterionic, and/or amphoteric surfactants, as well as mixtures thereof, can be preferred according to the present invention. Anionic surfactants are used in particular when the compositions according to the present invention are intended to be used as shower gels.

The surfactants (T) are used in quantities from 0.05 to 45 wt %, preferably 0.1 to 30 wt %, and very particularly preferably from 0.5 to 25 wt %, b based on the entire agent used according to the present invention.

Emulsifiers usable according to the present invention are, for example:
addition products of 4 to 30 mol ethylene oxide and/or 0 to 5 mol propylene oxide with linear fatty alcohols having 8 to 22 carbon atoms, with fatty acids having 12 to 22 carbon atoms, and with alkylphenols having 8 to 15 carbon atoms in the alkyl group,
C12 to C22 fatty acid mono- and diesters of addition products of 1 to 30 mol ethylene oxide with polyols having 3 to 6 carbon atoms, in particular with glycerol,
addition products of ethylene oxide and polyglycerol with methyl glucoside fatty acid esters, fatty acid alkanolamides, and fatty acid glucamides,
C8 to C22 alkyl mono- and oligoglycosides and ethoxylated analogs thereof, degrees of oligomerization from 1.1 to 5, in particular 1.2 to 2.0, and glucose as the sugar component, being preferred,
mixtures of alkyl(oligo)glucosides and fatty alcohols, for example the commercially available product Montanov® 68,
addition products of 5 to 60 mol ethylene oxide with castor oil and hardened castor oil,
partial esters of polyols having 3 to 6 carbon atoms with saturated fatty acids having 8 to 22 carbon atoms,
Sterols, for example cholesterol and lanosterol as zoosterols and ergosterol, stigmasterol, and sitosterol as phytosterols.
Phospholipids, e.g. lecithins or phosphatidylcholines from egg yolk or plant seeds (e.g. soybeans),
fatty acid esters of sugars and sugar alcohols, such as sorbitol,
polyglycerols and polyglycerol derivatives such as, for example, polyglycerol poly-12-hydroxystearate (commercial product Dehymuls® PGPH),
linear and branched fatty acids having 8 to 30 carbon atoms, and the Na, K, ammonium, Ca, Mg, and Zn salts thereof.

The agents according to the present invention contain the emulsifiers preferably in quantities from 0.1 to 25 wt %, in particular 0.5 to 15 wt %, based on the entire agent.

The compositions according to the present invention can preferably contain at least one nonionogenic emulsifier having an HLB value from 8 to 18. Nonionogenic emulsifiers having an HLB value from 10 to 15 can be particularly preferred according to the present invention.

The so-called “mild” emulsifiers are preferred emulsifiers according to the present invention.

The anionic polymers are anionic polymers that comprise carboxylate and/or sulfonate groups. Examples of anionic monomers of which such polymers can be made up are acrylic acid, methacrylic acid, crotonic acid, maleic acid anhydride, and 2-acrylamido-2-methylpropanesulfonic acid. The acid groups can be present entirely or partially as a sodium, potassium, ammonium, mono- or triethanolammonium salt. Preferred monomers are 2-acrylamido-2-methylpropanesulfonic acid and acrylic acid.

Anionic polymers that contain 2-acrylamido-2-methylpropanesulfonic acid as a sole monomer or co-monomer have proven to be very particularly effective, in which context the sulfonic acid group can be present entirely or partially as a sodium, potassium, ammonium, mono- or triethanolammonium salt.

The homopolymer of 2-acrylamido-2-methylpropanesulfonic acid that is available commercially, for example, under the designation Rheothik® 11-80, is particularly preferred.

Within this embodiment, it may be preferred to use copolymers of at least one anionic monomer and at least one nonionogenic monomer. With regard to the anionic monomers, reference is made to the substances listed above. Preferred nonionogenic monomers are acrylamide, methacrylamide, acrylic acid esters, methacrylic acid esters, vinylpyrrolidone, vinyl ethers, and vinyl esters.

Preferred anionic copolymers are acrylic acid/acrylamide copolymers and in particular polyacrylamide copolymers with sulfonic acid group-containing monomers. A particularly preferred anionic copolymer is made up of 70 to 55 mol % acrylamide and 30 to 45 mol % 2-acrylamido-2-methylpropanesulfonic acid, the sulfonic acid group being present entirely or partially as a sodium, potassium, ammonium, mono-, or triethanolammonium salt. This copolymer can also be present in crosslinked form, polyolefinically unsaturated compounds such as tetraallyoxyethane, allylsucrose, allylpentaerythritol, and methylene bisacrylamide preferably being used as crosslinking agents. One such polymer is contained in the commercial product Sepigel® 305 of the SEPPIC company. The utilization of this compound, which in addition to the polymer component contains a hydrocarbon mixture (C13 to C14 isoparaffin) and a nonionogenic emulsifier (Laureth-7), has proven particularly advantageous in the context of the teaching according to the present invention.

The sodium acryloyl dimethyl taurate copolymers marketed, under the designation Simulgel® 600, as a compound with isohexadecane and polysorbate-80, have also proven particularly effective according to the present invention.

Similarly preferred anionic homopolymers are uncrosslinked and crosslinked polyacrylic acids. Allyl ethers of pentaerythritol, of sucrose, and of propylene can be preferred crosslinking agents. Such compounds are obtainable commercially, for example, under the trademark Carbopol®.

Copolymers of maleic acid anhydride and methylvinyl ether, in particular those having crosslinks, are also color-preserving polymers. A maleic acid/methylvinyl ether copolymer crosslinked with 1,9-decadiene is obtainable commercially under the designation Stabileze® QM.

The anionic polymers are contained in the agents according to the present invention preferably in quantities from 0.05 to 10 wt %, based on the entire agent. Quantities from 0.1 to 5 wt % are particularly preferred.

In a further embodiment, the agents according to the present invention can contain nonionogenic polymers.

Suitable nonionogenic polymers are, for example:
Vinylpyrrolidone/vinyl ester copolymers such as those marketed, for example, under the trademark Luviskol® (BASF). Luviskol® VA 64 and Luviskol® VA 73, which are each vinylpyrrolidone/vinyl acetate copolymers, are likewise preferred nonionic polymers.
Cellulose ethers, such as hydroxypropyl cellulose, hydroxyethyl cellulose, and methylhydroxypropyl cellulose, such as those marketed, for example, under the trademarks Culminal® and Benecel® (AQUALON) and Natrosol® grades (Hercules).
Starch and derivatives thereof, in particular starch ethers, for example Structure® XL (National Starch), a multifunctional, salt-tolerant starch, shellac,
polyvinylpyrrolidones such as those marketed, for example, under the designation Luviskol® (BASF).

The nonionic polymers are contained in the compositions according to the present invention preferably in quantities from 0.05 to 10 wt %, based on the entire agent. Quantities from 0.1 to 5 wt % are particularly preferred.

The polymers (P) are contained in the compositions used according to the present invention preferably in quantities from 0.01 to 30 wt %, based on the entire composition. Quantities from 0.01 to 25, in particular from 0.01 to 15 wt %, are particularly preferred.

The following ingredients additionally contribute to the attainment and further enhancement of the effects according to the present invention.

With particular advantage, the compositions according to the present invention contain fatty substances (Fat) as a further active substance.

“Fatty substances” (Fat) are to be understood as fatty acids, fatty alcohols, natural and synthetic waxes, which can be present both in solid form and in liquid form in aqueous dispersion, and natural and synthetic cosmetic oil components.

The fatty acids (Fatac) that can be used are linear and/or branched, saturated and/or unsaturated fatty acids having 6 to 30 carbon atoms. Fatty acids having 10 to 22 carbon atoms are preferred. Among those that might be mentioned are, for example, the isostearic acids, such as the commercial products Emersol® 871 and Emersol® 875, and isopalmitic acids such as the commercial product Edenor® IP 95, as well as all other fatty acids marketed under the Edenor® commercial designations (Cognis). Further typical examples of such fatty acids are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid, and erucic acid, as well as industrial mixtures thereof. The fatty acid cuts that are obtainable from coconut oil or palm oil are usually particularly preferred; the use of stearic acid is, as a rule, particularly preferred.

The quantity used is 0.1 to 15 wt % based on the entire agent. The quantity is preferably 0.5 to 10 wt %, and quantities from 1 to 5 wt % can be very particularly advantageous.

Fatty alcohols (Fatal) that can be used are saturated, mono- or polyunsaturated, branched or unbranched fatty alcohols having C6 to C30, preferably C10 to C22, and very particularly preferably C12 to C22 carbon atoms. Usable in the context of the invention are, for example, decanol, octanol, octenol, dodecenol, decenol, octadienol, dodecadienol, decadienol, oleyl alcohol, erucyl alcohol, ricinol alcohol, stearyl alcohol, isostearyl alcohol, cetyl alcohol, lauryl alcohol, myristyl alcohol, arachidyl alcohol, capryl alcohol, caprinyl alcohol, linoleyl alcohol, linolenyl alcohol, and behenyl alcohol, as well as Guerbet alcohols thereof, this listing being intended to be exemplary and not limiting in nature. The fatty alcohols preferably derive, however, from natural fatty acids; it is usually possible to proceed from an extraction from the esters of the fatty acids by reduction. Also usable according to the present invention are those fatty alcohol cuts that are generated by the reduction of naturally occurring triglycerides such as beef tallow, palm oil, peanut oil, colza oil, cottonseed oil, soybean oil, sunflower oil, and linseed oil, or from fatty acid esters resulting from transesterification products thereof with corresponding alcohols, and thus represent a mixture of different fatty alcohols. Such substances are, for example, available commercially under the designations Stenol®, e.g. Stenol® 1618, or Lanette®, e.g. Lanette® 0, or Lorol®, e.g. Lorol® C8, Lorol® C14, Lorol® C18, Lorol® C₈₋₁₈, HD-Ocenol®, Crodacol®, e.g. Crodacol® CS, Novol®, Eutanol® G, Guerbitol® 16, Guerbitol® 18, Guerbitol® 20, Isofol® 12, Isofol® 16 Isofol® 24, Isofol® 36, Isocarb® 12, Isocarb® 16, or Isocarb® 24. It is of course also possible according to the present invention to use wool-wax alcohols such as those available commercially under the designations Corona®, White Swan®, Coronet®, or Fluilan®. The fatty alcohols are used in quantities from 0.1 to 30 wt % based on the entire preparation, preferably in quantities from 0.1 to 20 wt %.

Natural or synthetic waxes (Fatwax) that can be used according to the present invention are solid paraffins or isoparaffins, carnauba waxes, beeswaxes, candelilla waxes, ozocerites, ceresin, spermaceti, sunflower wax, fruit waxes such as, for example, apple wax or citrus wax, or microcrystalline waxes made from PE or PP. Such waxes are obtainable, for example, via Kahl & Co., Trittau.

The quantity used is 0.1 to 50 wt % based on the entire agent, preferably 0.1 to 20 wt %, and particularly preferably 0.1 to 15 wt % based on the entire agent.

The total quantity of oil and fat components in the agents according to the present invention is usually 0.5 to 7.5 wt %, based on the entire agent. Quantities from 0.5 to 35 wt % are preferred according to the present invention.

A further synergistic active substance according to the present invention in the compositions according to the present invention having the active-substance complex according to the present invention is protein hydrolysates and/or derivatives thereof (P).

The term “protein hydrolysates” is also understood according to the present invention as total hydrolysates as well as individual amino acids and derivatives thereof, as well as mixtures of different amino acids. Polymers constructed from amino acids and amino-acid derivatives are also understood according to the present invention under the term “protein hydrolysates”. Included among the latter are, for example, polyalanine, polyasparagine, polyserine, etc. Further examples of compounds usable according to the present invention are L-alanyl-L-proline, polyglycine, glycyl-L-glutamine, or D/L-methionine-5-methylsulfonium chloride. β-Amino acids and derivatives thereof, such as β-alanine, anthranilic acid, or hippuric acid, can of course also be used according to the present invention. The molecular weight of the protein hydrolysates usable according to the present invention is between 75 (the molecular weight of glycine) and 200,000; the molecular weight is preferably 75 to 50,000 dalton, and very particularly preferably 75 to 20,000 dalton.

The following are recited as examples of amino acids and derivatives thereof as protein hydrolysates according to the present invention: alanine, arginine, carnitine, creatine, cystathionine, cysteine, cystine, cystic acid, glycine, histidine, homocysteine, homoserine, isoleucine, lanthionine, leucine, lysine, methionine, norleucine, norvaline, ornithine, phenylalanine, proline, hydroxyproline, sarcosine, serine, threonine, tryptophan, thyronine, tyrosine, valine, aspartic acid, asparagine, glutamic acid, and glutamine. Preferred amino acids are alanine, arginine, glycine, histidine, lanthionine, leucine, lysine, proline, hydroxyproline, serine, and asparagine. It is very particularly preferred to use alanine, glycine, histidine, lysine, serine, and arginine. Glycine, histidine, lysine, and serine are used most preferably.

According to the present invention, protein hydrolysates of both vegetable and animal origin, or of marine or synthetic origin, can be used.

Animal protein hydrolysates are, for example, hydrolysates of elastin, collagen, keratin, silk, and milk protein, which can also be present in the form of salts. Such products are marketed, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), and Kerasol® (Croda).

Also preferred according to the present invention are vegetable protein hydrolysates such as, for example, soy, almond, pea, moring a, potato, and wheat protein hydrolysates. Such proteins are obtainable, for example, under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda), Crotein® (Croda), or Puricare® LS 9658 (Laboratoires Sérobiologiques).

Further protein hydrolysates preferred according to the present invention are, for example, collagen hydrolysates from fish or algae, and protein hydrolysates from mussels, or pearl hydrolysates.

Examples of pearl extracts usable according to the present invention are the commercial products Pearl Protein Extract BG® or Crodarom® Pearl.

The protein hydrolysates (Pro) are contained in the compositions in concentrations from 0.001 wt % to 20 wt %, by preference from 0.05 wt % to 15 wt %, and very particularly preferably in quantities from 0.05 wt % to 5 wt %.

The effect of the compositions according to the present invention can be further enhanced by means of a 2-pyrrolidinone-5-carboxylic acid and/or its derivatives (J). The sodium, potassium, calcium, magnesium or ammonium salts in which the ammonium ion carries, in addition to hydrogen, one to three C1 to C4 alkyl groups, are preferred. The sodium salt is very particularly preferred. The quantities used in the agents according to the present invention are 0.05 to 10 wt % based on the entire agent, particularly preferably 0.1 to 5, and in particular 0.1 to 3 wt %.

A further preferred group of ingredients of the compositions according to the present invention having the active-substance complex according to the present invention is vitamins, provitamins, or vitamin precursors.

Vitamins, provitamins, and vitamin precursors that are allocated to groups A, B, C, E, F, and H are particularly preferred in this context.

The group of substances referred to as “vitamin A” includes retinol (vitamin A1) as well as 3,4-didehydroretinol (vitamin A2). β-Carotene is the provitamin of retinol. Vitamin A components that are suitable according to the present invention are, for example, vitamin A acid and its esters, vitamin A aldehyde, and vitamin A alcohol, as well as esters thereof such as the palmitate and acetate. The agents according to the present invention contain the vitamin A component preferably in quantities from 0.05 to 1 wt % based on the entire preparation.

Members of the vitamin B group or vitamin B complex are, among others:
Vitamin B1 (thiamine)
Vitamin B2 (riboflavin)
Vitamin B3. The compounds nicotinic acid and nicotinic acid amide (niacinamide) are often listed under this designation. Nicotinic acid amide is preferred according to the present invention; it is contained in the agents used according to the present invention preferably in quantities from 0.05 to 1 wt % based on the entire agent.
Vitamin B5 (pantothenic acid, panthenol, and pantolactone). In the context of this group, panthenol and/or pantolactone are preferably used. Derivatives of panthenol that are usable according to the present invention are, in particular, the esters and ethers of panthenol as well as cationically derivatized panthenols. Individual representatives are, for example, panthenol triacetate, panthenol monoethyl ether and its monoacetate, and cationic panthenol derivatives. The aforesaid compounds of the vitamin B5 type are contained in the agents according to the present invention preferably in quantities from 0.05 to 10 wt % based on the entire agent. Quantities from 0.1 to 5 wt % are particularly preferred.
Vitamin B6 (pyridoxine as well as pyridoxamine and pyridoxal).

Vitamin C (ascorbic acid). Vitamin C is utilized in the agents according to the present invention preferably in quantities from 0.1 to 3 wt % based on the entire agent. Utilization in the form of the palmitic acid ester, the glucosides, or the phosphates can be preferred. Utilization in combination with tocopherols can likewise be preferred.

Vitamin E (tocopherols, in particular α-tocopherol). Tocopherol and its derivatives, which include in particular the esters such as the acetate, the nicotinate, the phosphate, and the succinate, are contained in the agents according to the present invention preferably in quantities from 0.05 to 1 wt % based on the entire agent.

Vitamin F. The term “vitamin F” is usually understood to mean essential fatty acids, in particular linoleic acid, linolenic acid, and arachidonic acid.

Vitamin H. This refers to the compound (3aS,4S,6aR)-2-oxohexahydrothienol[3,4-d]-imidazole-4-valeric acid, for which the trivial name “biotin” has, however, now become established. Biotin is contained in the agents according to the present invention preferably in quantities from 0.0001 to 1.0 wt %, in particular in quantities from 0.001 to 0.01 wt %.

The compositions according to the present invention preferably contain vitamins, provitamins, and vitamin precursors from groups A, B, E and H. Panthenol, pantolactone, pyridoxine and its derivatives, as well as nicotinic acid amide and biotin, are particularly preferred.

Lastly, the use of plant extracts (L) in the compositions according to the present invention yields further synergistic advantages.

Especially preferred according to the present invention are the extracts from green tea, oak bark, nettle, hamamelis, hops, henna, chamomile, burdock root, horsetail, hawthorn, linden blossom, almond, aloe vera, pine needles, horse chestnut, sandalwood, juniper, coconut, mango, apricot, lemon, wheat, kiwi fruit, melon, orange, grapefruit, salvia, rosemary, birch, mallow, valerian, lady's-smock, wild thyme, yarrow, thyme, lemon balm, restharrow, coltsfoot, hibiscus, meristem, ginseng, coffee, cocoa, moringa, and ginger root.

An extract obtainable from plants of the genus Echinacea is used as a further particularly preferred plant extract.

Preferred compositions according to the present invention contain purine and/or purine derivatives within narrower quantity ranges. In this context, cosmetic agents preferred according to the present invention are characterized in that in that they contain, based on their weight, 0.001 to 2.5 wt %, by preference 0.0025 to 1 wt %, particularly preferably 0.005 to 0.5 wt %, and in particular 0.01 to 0.1 wt % purine(s) and/or purine derivative(s).

Some representatives of purine, the purines, and the purine derivatives are particularly preferred according to the present invention. Cosmetic agents preferred according to the present invention are characterized in that they contain purine, adenine, guanine, uric acid, hypoxanthine, 6-purinethiol, 6-thioguanine, xanthine, caffeine, theobromine, or theophylline. Caffeine in particular has proven successful in hair-cosmetic formulations; it can be used, for example, in shampoos, conditioners, hair tonics, and lotions, by preference in quantities from 0.005 to 0.25 wt %, more preferably from 0.01 to 0.1 wt %, and in particular from 0.01 to 0.05 wt % (based in each case on the composition).

A further preferred active substance in the agents according to the present invention is taurine (2-aminoethanesulfonic acid). Preferred taurine derivatives are N-monomethyltaurine and N,N-dimethyltaurine. Further taurine derivatives for purposes of the present invention are taurocholic acid and hypotaurine.

Agents according to the present invention that contain, based on their weight, 0.0001 to 10.0 wt %, by preference 0.0005 to 5.0 wt %, particularly preferably 0.001 to 2.0 wt %, and in particular 0.001 to 1.0 wt % taurine and/or a derivative of taurine, are particularly preferred.

In addition, it may prove advantageous if penetration adjuvants and/or swelling agents (M) are contained in the agents according to the present invention. To be included thereamong are, for example, urea and urea derivatives, guanidine and derivatives thereof, arginine and derivatives thereof, water glass, imidazole and derivatives thereof, histidine and derivatives thereof, benzyl alcohol, glycerol, glycol and glycol ethers, propylene glycol and propylene glycol ethers, for example propylene glycol monoethyl ether, carbonates, hydrogencarbonates, diols and triols, and in particular 1,2-diols and 1,3-diols such as, for example, 1,2-propanediol, 1,2-pentanediol, 1,2-hexanediol, 1,2-dodecanediol, 1,3-propanediol, 1,6-hexanediol, 1,5-pentanediol, 1,4-butanediol.

A further group of very particular ingredients of the compositions according to the present invention is bioquinones. Ubiquinone, which is known as Coenzyme Q10, is particularly preferred according to the present invention.

The bioquinone(s) is/are in the agents, according to the present invention, based on their weight, in a quantity from 0.0000005 to 2%, preferably in a quantity from 0.000001 to 1%, and in particular in a quantity from 0.00001 to 0.5%.

A further particularly preferred group of ingredients in the cosmetic compositions according to the present invention is betaines. To be recited as examples of betaines of the formula that are particularly suitable according to the present invention are: carnitine, carnitine tartrate, carnitine magnesium citrate, acetylcarnitine, 3-O-lauroyl-L-carnitine hydrochloride, 3-O-octanoyl-L-carnitine hydrochloride, 3-O-palmitoyl-L-carnitine hydrochloride, taurine, taurine lysylate, taurine tartrate, taurine ornithate, lysyl taurine, and ornithyl taurine, betalains, 1,1-dimethylproline, hercynine (Nα,Nα,Nα-trimethyl-L-histidinium betaine), ergothioneine (thioneine, 2-mercapto-Nα,Nα,Nα-trimethyl-L-histidinium betaine), choline, choline chloride, choline bitartrate, choline dihydrogen citrate, and the compound (referred to in the literature as “betaine”) N,N,N-trimethylglycine. These mixed salts can be preferred according to the present invention.

The agents according to the present invention contain the betaines in quantities from 0.001 to 20 wt %, based on the entire agent. A concentration from 0.05 to 10 wt % is preferred.

The cosmetic compositions according to the present invention can furthermore contain pharmacologically effective substances in order to achieve specific effects such as, for example, an anti-dandruff effect or actions against acne.

These substances are contained in quantities from 0.01 to 10 wt %, based in each case on the entire composition.

Further ingredients that can be used together in cosmetic compositions are preservatives. The substance classes listed in Appendix 6, Part A and B of the European Cosmetics Ordinance are utilized as preservatives. Mild preservation is particularly preferred, ideally without the addition of typical preservatives.

In a further embodiment, the agents according to the present invention should additionally contain at least one UV light protection filter. UVB filters can be oil-soluble or water-soluble.

In addition, the cosmetic agents can contain further active substances, adjuvants, and additives such as, for example:
dimethylisosorbide and cyclodextrins,
solvents and solubilizers,
dyes for coloring the agent,
anti-dandruff active substances such as piroctone olamine, zinc omadine, and climbazole,
active substances such as bisabolol,
complexing agents such as EDTA, NTA, β-alaninediacetic acid, and phosphonic acids,
opacifiers such as styrene/PVP copolymers and styrene/acrylamide copolymers,
pearlescent agents such as ethylene glycol mono- and distearate, as well as PEG-3 distearate,
pigments,
propellants such as propane-butane mixtures, N20, dimethyl ether, CO2, and air.

With regard to further optional components and the quantities of those components that are used, reference is made expressly to the relevant manuals known to one skilled in the art.

As already mentioned, the excellent care-providing effect of the agents according to the present invention is especially significant in that it provides outstanding results even in the presence of oxidizing agents, for example in the context of oxidizing hair dyeing.

A second subject of the invention is therefore a method for treating hair, in which a cosmetic agent in accordance with Claim 1 is applied onto the hair and is rinsed out of the hair after a contact time.

The contact time is preferably a few seconds to 100 minutes, particularly preferably 1 to 50 minutes, and very particularly preferably 1 to 30 minutes.

The Examples that follow are intended to explain the subject matter of the present invention without, however, limiting it.

### Examples

Unless otherwise noted, all quantitative indications are parts by weight.

The following formulations were prepared utilizing known manufacturing methods.

Rinse

| | Sp-1 | Sp-2 | Sp-3 | Sp-4 |
|---|---|---|---|---|
| Cutina GMS-V | 0.3 | 0.3 | 0.2 | 0.2 |
| Dehyquart F 75 | 3.8 | 0.8 | 1.0 | 1.0 |
| Lanette O | 4.0 | 4.0 | 3.0 | 3.0 |
| Eumulgin B2 | 0.2 | 0.2 | 0.3 | 0.3 |
| Paraffinum Liquidum | 0.8 | 0.8 | | |
| Varisoft W 575 PG | | | 4.0 | 4.0 |
| Quaternium-91 | 3.5 | 4.0 | 2.5 | 3.0 |
| PPG-3 Benzyl Ether Myristate | 0.5 | 0.5 | 0.8 | 0.8 |
| Incroquat Behenyl HE | 2.0 | 2.0 | 2.0 | 2.0 |
| Isopropyl myristate | 1.0 | 1.0 | 1.0 | 1.0 |
| Tego Amid S 18 | 1.5 | 2.0 | 0.3 | 0.3 |
| Empigen CSC | 0.5 | 0.75 | 1.5 | 2.0 |
| Salcare SC 96 | 0.4 | 0.3 | 0.6 | 0.6 |
| Citric acid | | | 0.4 | 0.4 |
| D-panthenol | | | 0.2 | 0.2 |
| Ajidew NL 50 | | | 1.0 | 1.0 |
| Dehyquart A-CA | 3.0 | 3.0 | | |
| Lactic acid | 0.5 | 0.5 | | |
| Dicaprylyl carbonate | 0.1 | 0.5 | 0.5 | 0.1 |
| Phenoxyethanol | 0.4 | 0.4 | 0.3 | 0.3 |
| Litchiderm LS 9704 | 0.1 | 0.3 | 0.1 | 0.2 |
| Ectoin | 0.1 | 0.3 | 0.3 | 0.3 |
| Taurine | 0.1 | 0.3 | 0.5 | 0.3 |
| Extrapone White Tea GW | 0.1 | 0.3 | 0.1 | 0.2 |
| Water | to 100 | to 100 | to 100 | to 100 |

Hair Therapy

| | K-1 | K-2 | K-3 | K-4 |
|---|---|---|---|---|
| Synthalen K | 0.3 | 0.3 | 0.3 | 0.3 |
| Sepigel 305 | 3.0 | 3.0 | 3.0 | 3.0 |
| Sodium hydroxide 50% | 0.15 | 0.15 | 0.15 | 0.15 |
| Luviskol K 30 (powder) | 0.15 | 0.15 | 0.15 | 0.15 |
| Polymer JR 400 | 0.4 | 0.4 | 0.4 | 0.4 |
| Gafquat 755 N | 0.5 | 0.5 | 0.5 | 0.5 |
| Dehyquart F 75 | 0.6 | 0.6 | 0.6 | 0.6 |
| Quaternium-27 | 0.5 | 0.75 | 1.0 | 2.0 |
| Quaternium-91 | 1.0 | 1.5 | 2.0 | 4.0 |
| PPG-3 Benzyl Ether Maleate | 0.3 | 0.3 | 0.5 | 1.0 |
| Akypoquat 131 | 1.0 | 2.0 | 2.5 | 4.0 |
| Tego Amid S18 | 0.5 | 1.0 | 1.0 | 1.5 |
| Rewoquat RTM 50 | | | 1.0 | 0.5 |
| Ethanol 96% | 17.0 | 17.0 | 17.0 | 17.0 |
| D-panthenol, 75% | 0.2 | 0.2 | 0.2 | 0.2 |
| Nicotinic acid amide | 0.1 | 0.1 | 0.1 | 0.1 |
| Ajidew NL 50 | 1.0 | 1.0 | 1.0 | 1.0 |
| Dicaprylyl carbonate | 0.1 | 0.5 | 0.5 | 0.1 |
| Litchiderm LS 9704 | 0.1 | 0.2 | 0.3 | 0.5 |
| Ectoin | 0.1 | 0.2 | 0.3 | 0.5 |
| Taurine | 0.1 | 0.2 | 0.3 | 0.5 |
| Extrapone White Tea GW | 0.1 | 0.2 | 0.3 | 0.5 |
| Water | to 100 | to 100 | to 100 | to 100 |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair conditioning agent containing
a) at least 0.01 wt % of a quaternary imidazoline derivative having at least two long fat residues in accordance with the formula 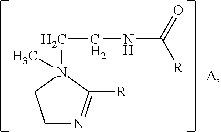
b) at least two ester oils,
c) a cosmetic carrier, and
d) no silicone.

2. The agent according to claim 1, wherein at least one of the ester oils is selected from the esters of carbonic acid with fatty alcohols.

3. The agent according to claim 2, wherein the esters of carbonic acid with the fatty alcohols are selected from the symmetrical esters of carbonic acid with fatty alcohols.

4. The agent according to claim 1, wherein at least one of the ester oils is selected from the ester oils of formula (D4-II)

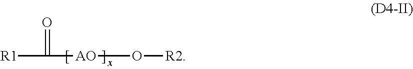

5. The agent according to claim 1, further comprising at least one further cationic compound.

6. The agent according to claim 5, wherein the further cationic compound is a cationic polymer.

7. The agent according to claim 1, further comprising at least one substance from the group of the vitamins, provitamins, and vitamin precursors as well as derivatives thereof, vitamins, provitamins, and vitamin precursors that are allocated to the groups A, B, C, E, F, and H being preferred.

8. A method of treating or cleaning skin or hair, comprising applying the preparation according to claim 1 to the skin or hair.

9. The method according to claim 8, further comprising rinsing the preparation after a contact time from 0 to 45 minutes.

10. The method according to claim 8, wherein the preparation is applied in an amount sufficient to restructure human hair.

11. The method according to claim 8, wherein the preparation is applied onto the skin and/or hair and is left there until the next washing.
